(19) United States
(12) Patent Application Publication
(10) Pub. No.: US 2024/0191277 A1
(43) Pub. Date: Jun. 13, 2024

(54) ACETYL-COA PRODUCING ENZYMES IN YEAST

(71) Applicant: DANISCO US INC., Palo Alto, CA (US)

(72) Inventors: Ulrike Maria Mueller, Echt (NL); Liang Wu, Echt (NL); Lourina Madeleine Raamsdonk, Echt (NL); Aaron Adriaan Winkler, Den Haag (NL)

(21) Appl. No.: 18/392,655

(22) Filed: Dec. 21, 2023

Related U.S. Application Data
(63) Continuation of application No. 17/081,187, filed on Oct. 27, 2020.
Application No. 17/081,187 is a continuation of application No. 14/045,683, filed on Oct. 3, 2013, now Pat. No. 10,927,399.
Application No. 14/045,683 is a continuation of application No. 12/670,050, filed on May 7, 2010, now abandoned.
(60) Provisional application No. 61/064,120, filed on Feb. 19, 2008.
(60) Provisional application No. 60/935,031, filed on Jul. 23, 2007.

(30) Foreign Application Priority Data
Jul. 23, 2007 (EP) 07112956.3
Dec. 21, 2007 (EP) 07123976.8
Feb. 19, 2008 (EP) 08101747.7

(51) Int. Cl. C12Q 1/32; C07K 14/33; C12N 9/02; C12P 7/16

(52) U.S. Cl. CPC C12Q 1/32; C07K 14/33; C12N 9/001; C12P 7/16; Y02E 50/10

(57) ABSTRACT

The present invention relates to a method of identifying a heterologous polypeptide having enzymatic activity for converting pyruvate, acetaldehyde or acetate into acetyl-CoA in (the cytosol of) of a yeast cell comprising: a) providing a mutated yeast cell comprising a deletion of at least one gene of the (PDH) by-pass, selected from the genes encoding the enzymes pyruvate decarboxylase (PDC), acetaldehyde dehydrogenase (ALD), and acetyl-CoA synthetase (ACS); b) transforming said mutated yeast cell with an expression vector comprising a heterologous nucleotide sequence encoding a candidate polypeptide having potential enzymatic activity for converting pyruvate, acetaldehyde or acetate into acetyl-CoA; c) testing said recombinant mutated yeast cell for its ability to grown on minimal medium containing glucose as sole carbon source, and d) identifying said candidate polypeptide as a heterologous polypeptide having enzymatic activity for converting pyruvate, acetaldehyde or acetate into acetyl-CoA in (the cytosol of) said yeast cell when growth of said cell is observed. The invention further relates to a method of producing a fermentation production such as butanol.

### CROSS REFERENCE TO RELATED APPLICATIONS

This application is a Continuation of U.S. application Ser. No. 14/045,683, filed Oct. 3, 2013, which is a Continuation of U.S. application Ser. No. 12/670,050, filed May 7, 2010, which is a § 371 National Stage Application of International Application No. PCT/EP2008/059119, filed Jul. 11, 2008, which claims priority to European Application No. 07112956.3, filed Jul. 23, 2007, U.S. Provisional Application No. 60/935,031, filed Jul. 23, 2007, European Application No. 07123976.8, filed Dec. 21, 2007, European Application No. 08101747.7, filed Feb. 19, 2008, and U.S. Provisional Application No. 61/064,120, filed Feb. 19, 2008, the content of all of which are incorporated herein by reference in their entireties.

### INCORPORATION BY REFERENCE OF THE SEQUENCE LISTING

The present application is being filed with a Sequence Listing in electronic format. The Sequence Listing is provided as an XML file entitled “IFF26480USPCN3_SequenceListing.xml,” created on Dec. 20, 2023, which is 43 KB in size. The information in the electronic format of the Sequence Listing is incorporated by reference in its entirety.

### BACKGROUND OF THE INVENTION

The present invention is in the field of metabolites production in yeast using heterologous expression systems. In particular, the present invention relates to the metabolic engineering of yeast strains capable of producing metabolites that require cytosolic acetyl-CoA as a precursor, such as butanol-producing yeast strains. The present invention relates to an assay system for identifying heterologous enzymes capable of converting pyruvate, acetaldehyde or acetate into cytosolic acetyl-CoA when expressed in the cytosol in yeast.

Acetyl-coenzyme A (CoA) is an essential intermediate in numerous metabolic pathways, and is a key precursor in the synthesis of many industrial relevant compounds, such as fatty acids, carotenoids, isoprenoids, vitamins, amino acids, lipids, wax esters, (poly)saccharides polyhydroxyalkanoates, statins, polyketides and acetic esters (such as ethyl acetate and isoamyl acetate). In particular, acetyl-CoA is also the precursor of the industrially important bulk chemical 1-butanol.

Compared to bacteria, such as E. coli, yeast cells provide a very suitable alternative to produce the above-mentioned acetyl-CoA derived products, in that yeast is not susceptible to phage or other infection since yeast-based processes may be run at low pH. Therefore, the use of yeast does not require a sterile process, thereby lowering the cost price of the product of interest.

When natural (wild type) yeast is not able to produce the acetyl-CoA-derived product of interest, the use of metabolic engineering can provide for yeast cells expressing heterologous genes that could support such a process. In such cases, the heterologous gene products are usually targeted to the cytosolic compartment of yeast. As the biosynthesis of acetyl-CoA-derived product will take place completely or partially in the cytosol, the supply of sufficient amounts of the precursor acetyl-CoA in the cytosolic compartment is crucial. In Saccharomyces cerevisiae, biosynthesis of acetyl-CoA takes place in two separate compartments. In mitochondria, acetyl-CoA is synthesized by oxidative decarboxylation of pyruvate catalyzed by the pyruvate dehydrogenase complex (PDH), with the following overall reaction stoichiometry:

Pyruvate(Pyr)+CoA+NAD+=acetyl-CoA+CO2+NADH+H+

In cytosol, acetyl-CoA is synthesized via the pyruvate dehydrogenase (PDH) by-pass, involving the enzymes pyruvate decarboxylase (PDC), acetaldehyde dehydrogenase (ALD), and acetyl-CoA synthetase (ACS), with the following overall reaction stoichiometry:

Pyr+CoA+ATP+NAD(P)+=acetyl-CoA+CO2+NAD(P)H+AMP+Ppi+H+.

Pyruvate-decarboxylase-negative (Pdc−) mutant of the yeast S. cerevisiae does not have a functional PDH by-pass, and cannot grow on minimal medium with glucose as the sole carbon source due to inability to supply (sufficient) cytosolic acetyl-CoA for growth (Flikweert et al., (1996) Yeast 12:247-57). The PDH by-pass is therefore essential in providing acetyl-CoA in the cytosolic compartment. However, the PDH bypass in yeast is not optimal with respect to the energy balance, as can be seen from the overall reaction stoichiometry: 2 moles of ATP are needed per acetyl-CoA synthesized via the PDH-bypass since in the acetyl-CoA synthetase reaction ATP is hydrolyzed to AMP. In contrast, the mitochondrial pathway via the PDH requires no ATP. The additional ATP requirement of the PDH by-pass can present a problem for synthesizing the product of interest from cytosolic acetyl-CoA precursor, as more carbon source needs to be diverted for ATP generation, via e.g. oxidative phosphorylation and/or substrate phosphorylation (e.g. glycolysis), thereby lowering the overall yield of the product on carbon.

When yeast is metabolically engineered to produce 1-butanol, heterologous biosynthetic genes of 1-butanol can be expressed in the cytosol in yeast cells (WO 2007/041269). In general 1 mole of glucose give rise to 2 moles of acetyl-CoA via glycolysis, which is the precursor of 1 mole of butanol; hence a maximum of 1 mole of butanol can be synthesized per mole of glucose if cell growth and maintenance is not considered. However, when the PDH by-pass is used in combination with butanol biosynthesis, this maximal theoretical yield cannot be achieved due to energy imbalance: whereas 2 moles of ATP are generated per mole of glucose converted in glycolysis, a total of 4 moles (2 times 2 mole) of ATP are needed in the PDH by-pass to form 2 moles of acetyl-CoA, which are converted to 1 mole of butanol. Thus, there is a net shortage of ATP if the PDH by-pass were used to synthesize 1 mole of 1-butanol from 1 mole of glucose.

Thus, there is a need for the identification of possible alternative metabolic routes for producing cytosolic acetyl-CoA in yeast, for the production of acetyl-CoA-derived products, in particular butanol, wherein the PDH by-pass is not required.

Butanol is an important industrial chemical and is suitable as an alternative engine fuel having improved properties over ethanol. Butanol also finds use as a solvent for a wide variety of chemical and textile processes, in the organic synthesis of plastics, as a chemical intermediate and as a solvent in the coating and food and flavor industry. Butanol can be produced from biomass (biobutanol) as well as fossil fuels (petrobutanol).

The chemical synthesis of butanol in one of its isomers can be accomplished by a variety of available methods known in the art (see e.g. Ullmann's Encyclopedia of Industrial Chemistry, 6th edition, 2003, Wiley-VCHVerlag GmbH and Co., Weinheim, Germany, Vol. 5. pp. 716-719). These processes have the disadvantage that they are based on the use of petrochemical derivates, are generally expensive, and are not environmentally friendly.

Biological synthesis of butanol can be achieved by fermentation using the acetone-butanol-ethanol (ABE) process carried out by the bacteria Clostridium acetobutylicum or other Clostridium species. An important disadvantage of the ABE process, however, is that it results in a mixture of acetone, 1-butanol and ethanol. Moreover, the use of bacteria requires sterile process conditions and generally renders the process susceptible to bacteriophage infection. Yeast cells thus provide a very suitable alternative as described above.

### SUMMARY OF THE INVENTION

The present inventors have now identified alternative metabolic routes for increasing the production of cytosolic acetyl-CoA in yeast which can overcome the problems of the PDH by-pass.

One possible route includes the direct conversion of acetaldehyde to acetyl-CoA without ATP consumption, by use of an acetylating acetaldehyde dehydrogenase (E.C. 1.2.1.10) (see FIG. 2, reaction A, ACDH). Another route includes the direct conversion of pyruvate to acetyl-CoA by an enzyme or a multi-enzyme-complex without ATP consumption, for instance, by use of a pyruvate:NADP oxidoreductase (E.C. 1.2.1.51) see FIG. 2, reaction C, PNO). In these two possible routes, the formation of 1 mole of butanol per mole of glucose would result in the formation of 2 moles of ATP. Yet another route includes the conversion of acetate to acetyl-CoA with 1 ATP consumed per acetyl-CoA formed by an alternative enzyme or a combination of enzymes, for instance, by use of acetate:CoA ligase (ADP-forming, E.C. 6.2.1.13), or by use of ATP:acetate phosphotransferase (E.C. 2.7.2.1) in combination with acetyl-CoA:Pi acetyltransferase (E.C. 2.3.1.8). In this route, the formation of 1 mole of butanol per mole of glucose is ATP-balanced, i.e. no ATP will be formed. The present inventors have now found that such an alternative to the PDH by-pass can result in acetyl-CoA synthesis in the cytosol of the yeast, and that such acetyl-CoA can be used biosynthetically to produce higher amounts of desirable fermentation products, such as butanol.

In a first aspect, the present invention provides a method of identifying a heterologous polypeptide having enzymatic activity for converting pyruvate, acetaldehyde or acetate into acetyl-CoA in (the cytosol of) a yeast cell comprising:
providing a mutated yeast cell, wherein said mutation comprises an inactivation of at least one gene of the (PDH) by-pass, selected from the genes encoding the enzymes pyruvate decarboxylase (PDC), acetaldehyde dehydrogenase (ALD), and acetyl-CoA synthetase (ACS);
transforming said mutated yeast cell with an expression vector comprising at least one heterologous nucleotide sequence operably linked to a promoter functional in yeast and said at least one heterologous nucleotide sequence encoding at least one candidate polypeptide having potential enzymatic activity for converting pyruvate, acetaldehyde or acetate into acetyl-CoA;
testing said recombinant mutated yeast cell for its ability to grow on minimal medium containing glucose as sole carbon source, and
identifying said candidate polypeptide as a heterologous polypeptide having enzymatic activity for converting pyruvate, acetaldehyde or acetate into acetyl-CoA in (the cytosol of) said yeast cell when growth of said cell is observed.

In a preferred embodiment of said method the yeast cell is a cell of Saccharomyces cerevisiae and the heterologous nucleotide sequence is codon (pair) optimized for expression in Saccharomyces cerevisiae.

In another preferred embodiment, said mutation comprises an inactivation of the gene for acetyl-CoA synthetase isoform 2 (acs2).

In another preferred embodiment, said at least one candidate polypeptide having enzymatic activity for converting acetaldehyde into acetyl-CoA is a (putative) acetylating acetaldehyde dehydrogenases.

Alternatively, said at least one heterologous polypeptide having enzymatic activity for converting pyruvate, acetaldehyde or acetate into acetyl-CoA in (the cytosol of) a yeast cell may consist of two or more enzymes working together to achieve the desired conversion from pyruvate, acetaldehyde or acetate into acetyl-CoA.

In another aspect, the present invention provides an integration vector for the integration in a yeast genome of a heterologous nucleotide sequence encoding a polypeptide having enzymatic activity for converting pyruvate, acetaldehyde or acetate into acetyl-CoA, and the subsequent expression of the heterologous polypeptide therefrom.

In another aspect, the present invention provides an expression vector expressing heterologous polypeptides in yeast, said expression vector comprising a heterologous nucleotide sequence operably linked to a promoter functional in yeast and said heterologous nucleotide sequence encoding a polypeptide having enzymatic activity for converting pyruvate, acetaldehyde or acetate into acetyl-CoA in (the cytosol of) said yeast cell.

In a preferred embodiment of said vector the polypeptide having enzymatic activity for converting pyruvate, acetaldehyde or acetate into acetyl-CoA is identified by a method according to the present invention as described above.

In another preferred embodiment, said polypeptide is selected from SEQ ID NO: 19, 22, 25, 28 and 52 and functional homologues thereof.

In another preferred embodiment, said expression vector is for expression in Saccharomyces cerevisiae, wherein said heterologous nucleotide sequence is codon (pair) optimized for expression in Saccharomyces cerevisiae.

In another preferred embodiment, said heterologous nucleotide sequence is selected from SEQ ID NO: 20, 23, 26 and 29.

In another aspect, the present invention provides a recombinant yeast cell comprising the expression vector of the present invention as described above.

In a preferred embodiment, the recombinant yeast cell further comprises an inactivation of at least one gene of the (PDH) by-pass, selected from the genes encoding the enzymes pyruvate decarboxylase (PDC), acetaldehyde dehydrogenase (ALD), and acetyl-CoA synthetase (ACS).

Preferably, a yeast cell according to be present invention comprises an inactivation of a gene encoding an acetyl-CoA synthase.

In another preferred embodiment, the recombinant yeast cell further comprises an inactivation of a gene (nucleotide sequence) encoding an enzyme capable of catalysing the conversion of acetaldehyde to ethanol, preferably a gene encoding an alcohol dehydrogenase.

As used herein, inactivation of a gene (nucleotide sequence) encoding an enzyme may be achieved by mutation, deletion or disruption of (part of) a gene or nucleotide sequence encoding an enzyme.

Preferably a yeast cell according to the present invention shows growth on minimal medium containing glucose as sole carbon source.

In another preferred embodiment of a yeast cell of the invention, said yeast cell further comprises one or more introduced genes encoding a recombinant pathway for the formation of 1-butanol from cytosolic acetyl-CoA. Suitable recombinant pathways from acetyl-CoA to 1-butanol are known in the art. Such pathways are for instance known from WO 2007/041269. Preferably said one or more introduced genes encode enzymes that produce acetoacetyl-CoA, 3-hydroxybutyryl-CoA, crotonyl-CoA, butyryl-CoA, butylaldehyde and/or 1-butanol. Said enzymes can be:
acetyl-CoA acetyltransferase (E.C. 2.3.1.9 [Enzyme Nomenclature 1992, Academic Press, San Diego]; although, enzyme's with a broader substrate range (E.C. 2.3.1.16) will be functional as well), which converts 2 moles of acetyl-CoA to acetoacetyl-CoA;
   NADH-dependent or NADPH-dependent 3-hydroxybutyryl-CoA dehydrogenase E.C. 1.1.1.35 or E.C. 1.1.1.30, resp. E.C. 1.1.1.157 or E.C. 1.1.1.36), which converts acetoacetyl-CoA to 3-hydroxybutyryl-CoA;
3-hydroxybutyryl-CoA dehydratase (also named crotonase; E.C. 4.2.1.17 or E.C. 4.2.1.55), which converts 3-hydroxybutyryl-CoA to crotonyl-CoA;
NADH-dependent or NADPH-dependent butyryl-CoA dehydrogenase (E.C. 1.3.1.44 resp. E.C. 1.3.1.38 or E.C.1.3.99.2), which converts crotonyl-CoA to butyryl-CoA;
monofunctional NADH-dependent or NADPH-dependent aldehyde dehydrogenase (E.C. 1.2.1.10, or 1.2.1.57), which converts butyryl-CoA to butyraldehyde, and
NADH-dependent or NADPH-dependent butanol dehydrogenase (E.C. 1.1.1.−), which converts butylaldehyde to 1-butanol, or
bifunctional NADH-dependent or NADPH-dependent aldehyde/alcohol dehydrogenase (E.C. 1.1.1.1.11.2.1.10), which converts butyryl-CoA to 1-butanol via butyraldehyde

In another preferred embodiment of the invention a yeast cell is a Saccharomyces cerevisiae.

In another aspect, the present invention provides a method of producing butanol, comprising the steps of fermenting a suitable carbon substrate with a yeast cell according to the present invention and recovering the butanol produced during said fermentation.

### BRIEF DESCRIPTION OF THE DRAWINGS

FIG. 1 is a schematic presentation of the PDH by-pass showing the enzymes pyruvate decarboxylase (PDC: E.C. 4.1.1.1), acetaldehyde dehydrogenase (ALD; E.C. 1.2.1.3, E.C. 1.2.1.4 and E.C. 1.2.1.5), and acetyl-CoA synthetase (ACS; E.C. 6.2.1.1).

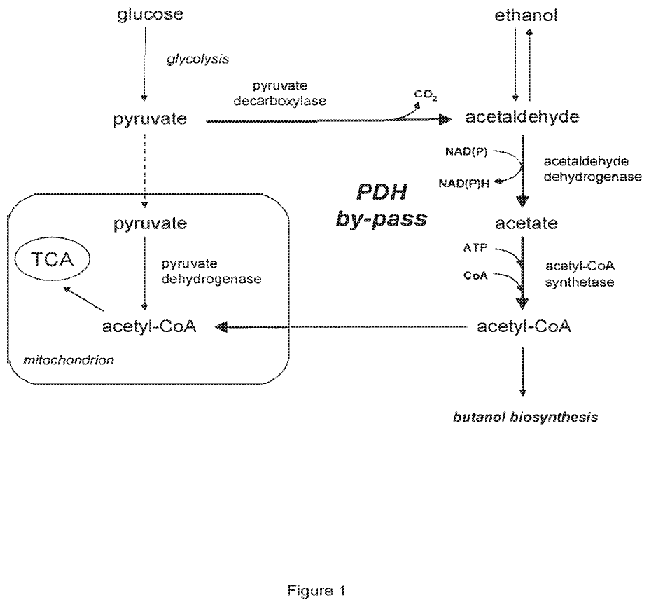

FIG. 2 shows a schematic metabolic route for butanol production in Saccharomyces cerevisiae. Reactions 1-6 are the butanol biosynthesis steps from Clostridium acetobutylicum introduced in yeast. A, B, and C indicate alternative reactions for acetyl-CoA biosynthesis in the cytosol. B indicates part of the pyruvate dehydrogenase by-pass (pdc, aid and acs), the natural source of cytosolic acetyl-CoA in yeast. Glc, glucose; EtOH, ethanol; Pyr, Pyruvate; AA, acetaldehyde; ACT, acetate; AcCoA, acetyl-CoA; AACOA, acetoacetyl-CoA; BuCoA, butyryl-CoA; Bual, butylaldehyde; BuOH, butanol; NAD(P)(H), nicotinamide adenine dinucleotide (phosphate) (in reduced form); ATP, adenosine triphosphate; AMP, adenosine monophosphate; TCA cycle, tricarboxylic acid cycle; PDH, pyruvate dehydrogenase; pdc, pyruvate decarboxylase; adh, alcohol dehydrogenase; acdh, acetylating acetaldehyde dehydrogenase; aid, acetaldehyde dehydrogenase; acs, acetyl-CoA synthetase; pno, pyruvate:NADP oxidoreductase. Enzymatic conversions indicated by reaction 1-6 indicate a heterologous butanol pathway from Clostridium acetobutylicum: thlB (or ThL) encoding acetyl-CoA acetyltransferase or thiolase [E.C. 2.3.1.9] (SEQ ID NO:30); hbd, 3-hydroxybutyryl-CoA dehydrogenase [E.C.1.1.1.157] (SEQ ID NO:31); crt, 3-hydroxybutyryl-CoA dehydratase [E.C.4.2.1.55] (SEQ ID NO:32); ter, trans-enoyl CoA reductase; bcd, butyryl-CoA dehydrogenase [E.C.1.3.99.2] (SEQ ID NO:33); etf αβ, heterodimeric electron transfer flavoprotein (elf α and etf β, SEQ ID NO:38 and SEQ ID NO:39, respectively); adhE/adhE1, aldehyde/alcohol dehydrogenase E and E1 [E.C. 1.1.1.1/1.2.1.10] (SEQ ID NO:34 and 35, respectively): bdhA/bdhB, NAD(P)H-dependent butanol dehydrogenase A and B [E.C.: 1.1.1.−] (SEQ ID NO:36 and 37, respectively).

FIG. 3 shows the map of plasmid YEplac112PtdhTadh. The sequence of this plasmid is provided in SEQ ID NO:40.

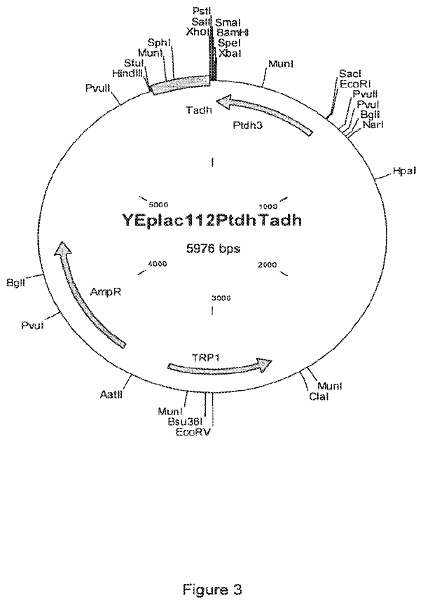

FIG. 4 shows an example of a similarity tree based on amino acid sequences of proteins of the types 1 to 4 as described in Example 2 and indicates the branches.

### DETAILED DESCRIPTION OF THE INVENTION

### Definitions

The term “butanol” refers to n-butanol, or 1-butanol.

The term “yeast” refers to a phylogenetically diverse group of single-celled fungi, most of which are in the division of Ascomycota and Basidiomycota. The budding yeasts (“true yeasts”) are classified in the order Saccharomycesales, with Saccharomyces cerevisiae as the most well known species.

The term “recombinant yeast” as used herein, is defined as a cell which contains a nucleotide sequence and/or protein, or is transformed or genetically modified with a nucleotide sequence that does not naturally occur in the yeast, or it contains additional copy or copies of an endogenous nucleic acid sequence (or protein), or it contains a mutation, deletion or disruption of an endogenous nucleic acid sequence.

The term “mutated” as used herein regarding proteins or polypeptides means that at least one amino acid in the wild-type or naturally occurring protein or polypeptide sequence has been replaced with a different amino acid, or deleted from the sequence via mutagenesis of nucleic acids encoding these amino acids. Mutagenesis is a well-known method in the art, and includes, for example, site-directed mutagenesis by means of PCR or via oligonucleotide-mediated mutagenesis as described in Sambrook et al., Molecular Cloning—A Laboratory Manual. 2nd ed., Vol. 1-3 (1989). The term “mutated” as used herein regarding genes means that at least one nucleotide in the nucleotide sequence of that gene or a regulatory sequence thereof, has been replaced with a different nucleotide, or has been deleted from the sequence via mutagenesis, resulting so in the transcription of a non-functional protein sequence or the knock-out of that gene.

The term “gene”, as used herein, refers to a nucleic acid sequence containing a template for a nucleic acid polymerase, in eukaryotes, RNA polymerase II. Genes are transcribed into mRNAs that are then translated into protein.

The term pyruvate dehydrogenase (PDH) by-pass refers to the enzymatic cascade form pyruvate to acetyl-CoA in the cytosol of yeast, and which consists of the following enzymes: pyruvate decarboxylase (PDC; E.C. 4.1.1.1) converting pyruvate into acetaldehyde; acetaldehyde dehydrogenase (ALD; E.C. 1.2.1.3, E.C. 1.2.1.4 and E.C. 1.2.1.5), converting acetaldehyde into acetate; and acetyl-CoA synthetase (ACS; E.C. 6.2.1.1), converting acetate into acetyl-CoA.

The term “nucleic acid” as used herein, includes reference to a deoxyribonucleotide or ribonucleotide polymer, i.e. a polynucleotide, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation.

Sequence identity is herein defined as a relationship between two or more amino acid (polypeptide or protein) sequences or two or more nucleic acid (polynucleotide) sequences, as determined by comparing the sequences. Usually, sequence identities are compared over the whole length of the sequences compared. In the art, “identity” also means the degree of sequence relatedness between amino acid or nucleic acid sequences, as the case may be, as determined by the match between strings of such sequences.

Preferred methods to determine identity are designed to give the largest match between the sequences tested. Methods to determine identity are codified in publicly available computer programs. Preferred computer program methods to determine identity and similarity between two sequences include BLASTP, BLASTN (Altschul, S. F. et al., J. Mol. Biol. 215:403-410 (1990), publicly available from NCBI and other sources (BLAST Manual, Altschul, S., et al., NCBI NLM NIH Bethesda, Md. 20894). Preferred parameters for amino acid sequences comparison using BLASTP are gap open 11.0, gap extend 1, Blosum 62 matrix.

Every nucleic acid sequence herein that encodes a polypeptide also, by reference to the genetic code, describes every possible silent variation of the nucleic acid. The term “conservatively modified variants” applies to both amino acid and nucleic acid sequences. With respect to particular nucleic acid sequences, conservatively modified variants refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation.

“Expression” refers to the transcription of a gene into structural RNA (rRNA, tRNA) or messenger RNA (mRNA) with subsequent translation into a protein.

As used herein, “heterologous” in reference to a nucleic acid or protein is a nucleic acid or protein that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

As used herein “promoter” is a DNA sequence that directs the transcription of a (structural) gene. Typically, a promoter is located in the 5′-region of a gene, proximal to the transcriptional start site of a (structural) gene. Promoter sequences may be constitutive, inducible or repressible. If a promoter is an inducible promoter, then the rate of transcription increases in response to an inducing agent.

The term “vector” as used herein, includes reference to an autosomal expression vector and to an integration vector used for integration into the chromosome.

The term “expression vector” refers to a DNA molecule, linear or circular, that comprises a segment encoding a polypeptide of interest under the control of (i.e., operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular an expression vector comprises a nucleotide sequence that comprises in the 5′ to 3′ direction and operably linked: (a) a yeast-recognized transcription and translation initiation region, (b) a coding sequence for a polypeptide of interest, and (c) a yeast-recognized transcription and translation termination region. “Plasmid” refers to autonomously replicating extrachromosomal DNA which is not integrated into a microorganism's genome and is usually circular in nature.

An “integration vector” refers to a DNA molecule, linear or circular, that can be incorporated in a microorganism's genome and provides for stable inheritance of a gene encoding a polypeptide of interest. The integration vector generally comprises one or more segments comprising a gene sequence encoding a polypeptide of interest under the control of (i.e., operably linked to) additional nucleic acid segments that provide for its transcription. Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is nonfunctional in that organism. Integration of the segment comprising the gene of interest may be selected if an appropriate marker is included within that segment.

As used herein, the term “operably linked” refers to a juxtaposition wherein the components so described are in a relationship permitting them to function in their intended manner. A control sequence “operably linked” to another control sequence and/or to a coding sequence is ligated in such a way that transcription and/or expression of the coding sequence is achieved under conditions compatible with the control sequence. Generally, operably linked means that the nucleic acid sequences being linked are contiguous and, where necessary to join two protein coding regions, contiguous and in the same reading frame.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are cells of the order of Actinomycetales, most preferably yeast cells, most preferably cells of Saccharomyces cerevisiae.

“Transformation” and “transforming”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host cell genome.

The term “oligonucleotide” refers to a short sequence of nucleotide monomers (usually 6 to 100 nucleotides) joined by phosphorous linkages (e.g., phosphodiester, alkyl and aryl-phosphate, phosphorothioate, phosphotliester), or non-phosphorous linkages (e.g., peptide, sulfamate and others). An oligonucleotide may contain modified nucleotides having modified bases (e.g., 5-methyl cytosine) and modified sugar groups (e.g., 2′-O-methyl ribosyl, 2′-O-methoxyethyl ribosyl, 2′-fluoro ribosyl, 2′-amino ribosyl, and the like). Oligonucleotides may be naturally-occurring or synthetic molecules of double- and single-stranded DNA and double- and single-stranded RNA with circular, branched or linear shapes and optionally including domains capable of forming stable secondary structures (e.g., stem-and-loop and loop-stem-loop structures).

The term “polynucleotide” as used herein refers to a polymeric form of nucleotides of any length, either ribonucleotides or deoxyribonucleotides. Thus, this term includes double- and single-stranded DNA and RNA.

The term “recombinant polynucleotide” as used herein intends a polynucleotide of genomic, cDNA, semisynthetic, or synthetic origin which, by virtue of its origin or manipulation: (1) is not associated with all or a portion of a polynucleotide with which it is associated in nature; or (2) is linked to a polynucleotide other than that to which it is linked in nature; or (3) does not occur in nature.

The term “minimal medium” as used herein refers to a chemically defined medium, which includes only the nutrients that are required by the cells to survive and proliferate in culture. Typically, minimal medium is free of biological extracts, e. g., growth factors, serum, pituitary extract, or other substances, which are not necessary to support the survival and proliferation of a cell population in culture. For example, minimal medium generally includes as essential substances: at least one carbon source, such as glucose; at least one nitrogen source, such as ammonium, ammonium sulfate, ammonium chloride, ammonium nitrate or urea; inorganic salts, such as dipotassium hydrogenphosphate, potassium dihydrogen-phosphate and magnesium sulfate; and other nutrients, such as biotin and vitamins.

### DESCRIPTION OF THE PREFERRED EMBODIMENTS

A method of the present invention provides a method for identifying heterologous enzymes capable of producing acetyl-CoA in the cytosol of a yeast cell.

The heterologous enzyme may produce the acetyl-CoA using pyruvate, acetaldehyde or acetate as a substrate, preferably in a single conversion step. Preferably, the heterologous enzyme produces the acetyl-CoA from acetaldehyde. An enzyme capable of catalyzing said reaction is acetylating acetaldehyde dehydrogenase (acdh; E.C. 1.2.1.10) also referred to as acetadehyde:NAD+ oxidoreductase (CoA-acetylating). The conversion of acetaldehyde into acetyl-CoA by acetylating acetaldehyde dehydrogenase is reversible and runs in the direction of acetyl-CoA when acetaldehyde accumulates in the cytosol. Such an accumulation may for instance be achieved by deletion of alcohol dehydrogenase (adh: E.C. 1.1.1.1).

The heterologous enzyme may also produce the acetyl-CoA from pyruvate. An enzyme capable of catalyzing said reaction is a pyruvate:NADP oxidoreductase (pno; E.C. 1.2.1.51). The reaction is stoichiometrically identical to the mitochondrial pyruvate dehydrogenase except that pno uses NADPH as a cofactor as compared to PDH that uses NADH. Compared to acdh, an important disadvantage of the pno enzyme system is that pno is oxygen sensitive, and that it is a large multimeric enzyme, and hence, its successful genetic incorporation (a 5-6 kb gene) is much more difficult than that of acdh. For this reason, the use of acdh is preferred in embodiments of the present invention.

An important feature of a test cell capable of revealing the desired enzymatic activity of a test polypeptide is that the cell is prototrophic as a result of the introduced polypeptide. With this, it is meant that the cell's nutritional requirements do not exceed those of the corresponding wild-type strain and that it will proliferate on minimal medium (in contrast to the auxotroph). In fact, the production of acetyl-CoA as supported by the test polypeptide will cancel the effect of the deletion of said at least one gene of the PDH by-pass, caused by the deletion of the gene for pyruvate decarboxylase (pdc; E.C. 4.1.1.1), acetaldehyde dehydrogenase (aid; E.C. 1.2.1.3. E.C. 1.2.1.4 or E.C. 1.2.1.5), or acetyl-CoA synthetase (acs; E.C. 6.2.1.1). Such complementation assays are well known in the art. In aspects of the present invention the assay is used to identify suitable sources of heterologous enzymes capable of sustaining cytosolic acetyl-CoA production in yeast cells.

The complementation assay is based on the provision of alternative routes to overcome the deleted enzyme activity of the PDH by-pass. Methods for effecting deletion of genes in yeast are well known in the art, and can for instance be achieved by oligonucleotide-mediated mutagenesis. Good results may be obtained with the plasmid pUG6 carrying the loxP-kanMX-IoxP gene disruption cassette (Güldener et al. Nucleic Acids Res. 24(13):2519-24; GenPept accession no. P30114). Thus, the skilled person will be able to provide a yeast strain having a deleted acetaldehyde dehydrogenase and/or acetyl-CoA synthetase gene for blocking the PDH by-pass therein.

Saccharomyces cerevisiae comprises two acetyl-CoA synthetase isoforms, Acs1p and Acs2p. Both are the nuclear source of acetyl-CoA for histone acetylation. The production of cytosolic acetyl-CoA is also required for lipid production. Acs activity is essential, since an acs1 acs2 double null mutant is non-viable. An acs1 null mutant can grow with ethanol as the sole carbon source. The mutated yeast cell used in aspects of the present invention preferably has an inactivation of the acs2 gene.

Saccharomyces cerevisiae mutants carrying an inactivation of the acs2 gene are not able to grow on glucose as sole carbon source, because ACS1 is repressed and the protein is actively degraded. Complementation of such a delta acs2 mutant with a plasmid based acs gene will restore the cell's ability to grow on glucose as single carbon source. In addition, growth of such a mutant is complemented by the expression of genes supporting alternative routes for the production of sufficient cytosolic acetyl-CoA. Thus, transformation of the delta acs2 mutant with a plasmid from which a functional (heterologous) acdh or pno can be expressed will restore the mutant's ability to grow on glucose as sole carbon source. It should be understood that in addition to the removal of the ACS2 locus, one may also remove the ACS1 locus. Although it is believed that this may in some instances prevent the occurrence of revertants (mutations in the ACS1 locus leading to reversion of the delta acs2 phenotype), this was however not found to be essential. Double mutants (acs1/acs2Δ strains) would be wholly dependent on the introduced acdh or pno gene for the production of cytosolic acetyl-CoA.

An important advantage of a complementation assay of the present invention is that it can be performed as a plate screening assay wherein successful complementation is observed as colony growth. This is much faster than experiments that require the analysis for the production of a desired metabolic product.

For complementation of the mutation, the yeast cell having the inactivated aid and/or acs gene is then transformed with a suitable expression vector comprising a nucleotide sequence of a heterologous test polypeptide.

Yeast expression vectors are widely available from a variety of commercial suppliers. To date, functional complementation of yeast mutations by foreign homologues has become a standard practice in engineering of Saccharomyces cerevisiae. Suitable expression vectors for heterologous gene expression may be based on artificial, inducible promoters such as the GAL promoter, but is preferably based on constitutive promotors such as the TDH3 promoter. Suitable systems are exemplified in the examples below. In certain production systems, the use of an inducible promotor may be preferred, as it would allow for temporal separation of stages for biomass production (promotor not induced) and fermentation product production (promoter induced). In another highly preferred embodiment in certain production systems, the vector is in integration vector for stable integrating the heterologous genes in the genome of the yeast production strain.

In order to achieve optimal expression in yeast, the codon (pair) usage of the heterologous gene may be optimized by using any one of a variety of synthetic gene design software packages, for instance GeneOptimizer® from Geneart AG (Regensburg, Germany) for codon usage optimization or codon pair usage optimization as described in WO2008/000632. Such adaptation of codon usage ensures that the heterologous genes, which are for instance of bacterial origin, are effectively processed by the yeast transcription and translation machinery. Optimization of codon pair usage will result in enhanced protein expression in the yeast cell.

The optimized sequences may for instance be cloned into a high copy yeast expression plasmid, operably linked to a (preferably constitutive) promoter functional in yeast. Good results have been obtained with the plasmid YEplac112 (2μ TRP1) (Gietz & Sugino Gene 74(2):527-34).

Heterologous genes that encode a candidate polypeptide having potential enzymatic activity for converting pyruvate, acetaldehyde or acetate into acetyl-CoA may be identified in silico. Suitable enzymes described as possessing the capacity to convert acetaldehyde into acetyl-CoA are acetylating acetaldehyde dehydrogenases (E.C. 1.2.1.10). The nucleotide and amino acid sequences of over 200 of these enzymes from a variety of microbial origins are described in various databases (e.g. the KEGG (Kyoto Encyclopedia of Genes and Genomes) database).

The present inventors have selected several acetylating acetaldehyde dehydrogenases and tested these in the delta acs2 mutant-based assay system of the present invention. Many of these, though not all, were functional in S. cerevisiae when codon pair usage was optimized.

Functional homologues to these proteins can also be used in aspects of the present invention. The term “functional homologues” as used herein refers to a protein comprising the amino acid sequence of SEQ ID NO: 19, 22, 25 or the acetaldehyde dehydrogenase part of SEQ ID NOs: 28 and 52 in which one or more amino adds are substituted, deleted, added, and/or inserted, and which protein has the same enzymatic functionality for substrate conversion, for instance an acetylating acetaldehyde dehydrogenase homologue is capable of converting acetaldehyde into acetyl-CoA. This functionality may be tested by use of an assay system comprising a recombinant yeast cell comprising an expression vector for the expression of the homologue in yeast, said expression vector comprising a heterologous nucleotide sequence operably linked to a promoter functional in yeast and said heterologous nucleotide sequence encoding the homologous polypeptide of which enzymatic activity for converting pyruvate, acetaldehyde or acetate into acetyl CoA in (the cytosol of) said yeast cell is to be tested, and performing a method for identifying a heterologous polypeptide having enzymatic activity for converting pyruvate, acetaldehyde or acetate into acetyl-CoA in (the cytosol of) a yeast cell as described herein using said assay system. Candidate homologues may be identified by using in silico similarity analyses. A detailed example of such an analysis is described in Example 2 below. The skilled person will be able to derive therefrom how suitable candidate homologues may be found and, optionally upon codon(pair) optimization, will be able to test the required functionality of such candidate homologues using the assay system of the present invention as described above. A suitable homologue represents a polypeptide having an amino acid sequence identity to an acetylating acetaldehyde dehydrogenase of more than 50%, preferably more than 60%, more preferably more than 70%, 80%, 90% or more, for instance having such an amino acid sequence identity to SEQ ID NOs: 19, 22, 25, or the acetaldehyde dehydrogenase part of SEQ ID NOs:28 and 52 and having the required enzymatic functionality for converting acetaldehyde into acetyl-CoA. Similarly, enzymes described for the direct conversion of pyruvate into acetyl-CoA and the functional homologues thereof, as well as enzymes described for the conversion of acetate to acetyl-CoA and the functional homologues thereof, can also be used, similar as described for acetylating acetaldehyde dehydrogenase above.

A method of the present invention further comprises the step of testing the ability of the mutated and test-protein transformed yeast cell to grow on minimal medium containing glucose as sole carbon source. As stated earlier, this may suitably occur on solid (agar) media in Petri dishes (plates) where growth can be observed as growth of a colony, however, liquid media are equally suitable and growth may be detected by turbidity. Other methods for determining growth of the mutated and test-protein transformed yeast cell on minimal medium containing glucose as sole carbon source may also be used.

When the mutated and test-protein-transformed yeast cell is capable of growth on minimal medium with glucose, the candidate polypeptide is successfully identified as a heterologous polypeptide having enzymatic activity for converting pyruvate, acetaldehyde or acetate into acetyl-CoA in (the cytosol of) said yeast cell. Growth may suitably be observed as colony formation on solid growth media, in particular minimal medium containing glucose.

An expression vector for the expression of heterologous polypeptides in yeast, according to the present invention may be any expression vector suitable for transforming yeast. Innumerable examples are available in the art that can suitably be used to express heterologous nucleotide sequences in yeast. A very suitable vector in aspects of the invention is a plasmid. A highly preferred plasmid is YEplac112PtdhTadh (SEQ ID NO:40).

Generally, the heterologous nucleotide sequence encoding the polypeptide having enzymatic activity for converting pyruvate, acetaldehyde or acetate into acetyl CoA in (the cytosol of) said yeast cell, will be placed under control of a promoter functional in yeast. Preferably the promoter is a constitutive promoter. The promoter on plasmid YEplac112PtdhTadh is the TDH3 promoter.

The heterologous nucleotide sequences incorporated in the expression vector of the present invention may be any pno, acdh or other enzyme capable of converting pyruvate, acetaldehyde or acetate (respectively) into acetyl-CoA in the cytosol of the yeast. Preferred nucleotide sequences are those as identified herein, namely the nucleotide sequences encoding:
the ethanolamine utilization protein EutE from E. coli HS (nucleotide sequences with SEQ ID NO:18);
the hypothetical protein Lin1129 from Listeria innocua similar to ethanolamine utilization protein EutE. (nucleotide sequences with SEQ ID NO:21)
the acetaldehyde dehydrogenase EDK33116 from Clostridium kluyver DSM 555 (nucleotide sequences with SEQ ID NO:24); and
the adhE homologue of S. aureus (nucleotide sequences with SEQ ID NO:27) encoding a bifunctional acetaldehyde/alcohol dehydrogenase in Staphylococcus aureus subsp. aureus N315, or the acetaldehyde dehydrogenase functional part thereof.
the adhE homologue of Piromyces sp. E2 (nucleotide sequence SEQ ID NO: 51) encoding a bifunctional acetaldehyde/alcohol dehydrogenase, or the acetaldehyde dehydrogenase part thereof.

Also suitable are functional homologues of these nucleotide sequences, or of the polypeptides that they encode. With this term is meant that a nucleic acid sequence having more than 80%, 90% or 95% sequence identity with the nucleotide sequences encoding the above acdh enzymes, or having more than 50%, preferably more than 60%, 70%, 80%, 90%, or 95% sequence identity with the amino acid sequence of the above acdh enzymes, with the proviso that the polypeptides encoded by the homologous sequences exhibit functional enzymatic acdh activity.

As stated above, these nucleotide sequences can be optimized for expression in Saccharomyces cerevisiae by optimization of codon pair usage well known in the art. Codon pair optimized sequences for the SEQ ID NO:18, 21, 24, and 27 are provided in SEQ ID NO20, 23, 26, and 29, respectively.

The expression vector of the invention may be used to transform a yeast cell. Methods of transformation include electroporation, glass bead and biolistic a transformation, all of which are well known in the art and for instance described in Sambrook et al., Molecular Cloning-A Laboratory Manual, 2nd ed., Vol. 1-3 (1989).

A yeast cell according to the present invention comprises a heterologous nucleotide sequence encoding a polypeptide having enzymatic activity for converting pyruvate, acetaldehyde or acetate into acetyl-CoA in (the cytosol of) said yeast cell. Preferably, a yeast cell of the invention comprises a heterologous acdh or pno. The advantage of such a yeast cell is that it can produce acetyl-CoA by a metabolic route wherein the PDH by-pass is not required. This is energetically more favourable under anaerobic conditions, and may form the basis of any biological synthesis process using yeast cells under anaerobic conditions where acetyl-CoA is an intermediate. In addition to comprising the heterologous acdh or pno, the yeast cell of the invention may comprise various gene deletions or gene supplementations, depending on the intended use of the yeast.

Preferably a yeast cell according to the present invention comprises an inactivation of a nucleotide sequence (gene) encoding an enzyme capable of catalysing the conversion of acetaldehyde to ethanol, preferably an alcohol dehydrogenase, for instance to optimize acetaldehyde accumulation in the yeast cell.

If used in a method of screening for heterologous enzymes according to a method of the invention, the yeast cell comprises a deletion of at least one gene of the (PDH) by-pass, selected from the genes encoding the enzymes pyruvate decarboxylase (PDC), acetaldehyde dehydrogenase (ALD), and acetyl-CoA synthetase (ACS), preferably acetyl-CoA synthetase, most preferably acs2.

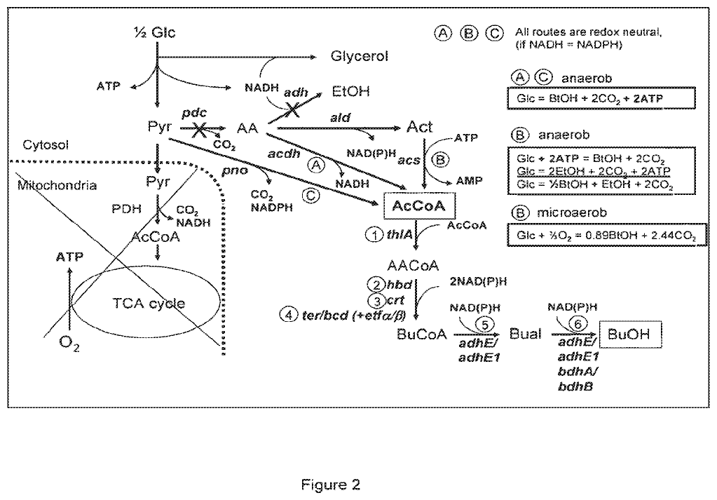

If used in a method of producing a fermentation product, the yeast cell may optionally comprise a number of (heterologous) gene supplementations supporting the metabolic pathway from acetyl-CoA to said butanol. Such a pathway may consist only of heterologous gene products, or may make use of a mixture of heterologous and endogenous gene products. In the event the fermentation product is butanol, use can be made of a yeast comprising genes encoding enzymes for the butanol pathway of e.g. Clostridium acetobutylicum as described herein and in FIG. 2. In the event the yeast cell according to the present invention comprises genes encoding enzymes for butanol production, the yeast preferably comprises a nucleotide sequence encoding a butyryl-CoA dehydrogenase and at least one nucleotide sequence encoding a heterologous electron transfer flavoprotein (ETF). It was found that a yeast cell comprising an ETF in addition to genes of the butanol pathway produces an increased amount of butanol.

A heterologous electron transfer flavoprotein in the eukaryotic cell according to the present invention may be a single protein or the ETF may comprise two or more subunits, for instance an alpha and a beta subunit. Preferably the ETF comprises an ETF alpha (SEQ ID NO: 38) and an ETF beta (SEQ ID NO: 39). The electron transfer flavoprotein may be derived from any suitable origin. Preferably, the ETF is derived from the same origin as the butyryl-CoA dehydrogenase. Preferably, the ETF is derived from prokaryotic origin preferably from a Clostridium sp., preferably a Clostridium acetobutylicum or a Clostridium beijerinckii.

A method for producing a fermentation product according to the present invention, preferably comprises growing a yeast under anaerobic conditions on a suitable carbon and energy source. Suitable sources of carbon and energy are C5 and C6 sugars (monosaccharides) such as glucose and polysaccharides such as starch. Other raw materials such as sugarcane, maize, wheat, barley, sugarbeets, rapeseed, and sunflower are also suitable. In some instances the raw material may be pre-digested by enzymatic treatment. Most preferably the carbon source is lignocellulose, which is composed of mainly cellulose, hemicellulose, pectin, and lignin. Lignocellulose is found, for example, in the stems, leaves, hulls, husks, and cobs of plants. Hydrolysis of these polymers by specific enzymatic treatment releases a mixture of neutral sugars including glucose, xylose, mannose, galactose, and arabinose. Lignocellulosic materials, such as wood, herbaceous material, agricultural residues, corn fiber, waste paper, pulp and paper mill residues can be used to produce butanol. Hydrolysing enzymes are for instance beta-linked glucans for the hydrolysis of cellulose (these enzymes include endoglucanases, cellobiohydrolases, glucohydrolases and beta-glucosidases); beta-glucosidases hydrolyze cellobiose; endo-acting and exo-acting hemicellulases and cellobiases for hydrolysis of hemicellulose, and acetylesterases and esterases that hydrolyze lignin glycoside bonds. These and other methods for hydrolysis of lignocellulose are well known in the art.

Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

The invention will now be illustrated by way of the following non-limiting examples.

### EXAMPLES

The following examples illustrate the provision of a strain of Saccharomyces cerevisiae useful in assays and methods of the present invention, for instance in methods for identifying heterologous enzymes capable of forming cytosolic acetyl-CoA in S. cerevisiae. Such methods are useful in the identification of routes/enzymes which allow the cytosolic supply of acetyl-CoA in S. cerevisiae under anaerobic conditions.

In order to enhance cytosolic acetyl-CoA formation in our butanol production strain, a selection method was set up to identify heterologous enzymes forming cytosolic acetyl-CoA in S. cerevisiae. The test system is based on a delta acs2 yeast mutant deficient in cytosolic acetyl-CoA biosynthesis on glucose, such a strain is unable to grow on glucose as sole carbon source unless cytosolic acetyl-CoA formation is complemented. Complementation studies in such a strain can reveal which heterologous enzymes are suitable for use in butanol producing strains of Saccharomyces cerevisiae.

Acetylating acetaldehyde dehydrogenase was identified to be a good candidate for cytosolic acetyl-CoA supply over the homologous PDH by-pass because no ATP is dissipated. Twelve putative acetylating acetaldehyde dehydrogenases, identified based on sequence homology, were synthesized and checked for complementation of the delta acs2 yeast.

The codon pair optimized genes of the eutE homologues of E. coli, L. innocua and C. kluyveri and the adhE homologue of S. aureus were able to complement the acs2 yeast mutants (4 out of 7), resulting in growth of the acs24 S. cerevisiae host. The aim is to improve butanol biosynthesis in yeast by expression of one or more genes so identified.

In order to test if these heterologous routes for cytosolic acetyl-CoA supply work in S. cerevisiae, a screening system was developed based on Saccharomyces cerevisiae mutants carrying a deletion of the acs2 gene. These cells are not able to grow on glucose as sole carbon source unless the delta acs2 mutant is complemented with a plasmid based acs gene or complemented with the expression of any other gene generating sufficient cytosolic acetyl-CoA. So if it were to be transformed with a plasmid leading to active expression of acdh or pno, such a mutant should be able to grow again with glucose as single carbon source. The complementation studies were performed on plates. The following experiments were performed to set up and evaluate the test system.

### Example 1

### Construction of Delta Acs2 Strain

The S. cerevsiae acs2 deleted strain (acs24 strain) was produced by first performing a PCR on plasmid pUG6 (Güldener et al., 1996. supra) with the following oligonucleotides:

5′acs2::Kanlox
5′-tacacaaacagaatacaggaaagtaaatcaatacaataataaaac
agctgaagcttcgtacgc-3′
3′acs2::Kanlox
5′-tctcattacgaaatttttctcatttaagttatttctttttttgag
gcataggccactagtggatctg-3′.

The resulting 1.4 kb fragment, containing the KanMX marker which confers resistance to G418, was used to transform S. cerevisiae CEN.PK113-3C (MATA trp1-289). After transformation the strain was plated on YPD (10 g l⁻¹ yeast extract (BD Difco), 20 g l⁻¹ peptone (BD Difco)), 10 g 1-1 glucose) with 200 mg/ml Geneticin (G418). In resistant transformants, correct integration was verified by PCR using oligonucleotides:

5′ACS2:
5′-gatattcggtagccqattcc-3′
3′ACS2:
5′-ccgtaaccttctcgtaatgc-3′
ACS2internal:
5′-cggattcgtcatcagcttca-3′
KanA:
5′-cgcacgtcaagactgtcaag-3′
KanB:
5′-tcgtatgtgaatgctggtcg-3′

The phenotype was verified by testing for growth on YP with 1% glucose (YPD) or 1% ethanol+1% glycerol (YPEG) as the carbon source.

One transformant that had the correct PCR bands and did not grow on YP with glucose, but did grow on with YP with ethanol and glycerol as the carbon sources, was picked and named RWB060 (MATA trp1-289 acs2::Kanlox).

### Example 2

### In Silico Identification of Putative Acetylating Acetaldehyde Dehydrogenases for Direct Conversion of Acetaldehyde to Acetyl-CoA

Enzymes described for the conversion of acetaldehyde to acetyl-CoA are the so-called acetylating acetaldehyde dehydrogenases (ACDH) (E.C. 1.2.1.10) catalysing the following reaction:

Acetaldehyde (AA)+NAD⁺+CoA<=>Acetyl-CoA+NADH+H+

From literature four types of proteins have been described that have this activity:

1) Bifunctional proteins that catalyze the reversible conversion of acetyl-CoA to acetaldehyde, and the subsequent reversible conversion of acetaldehyde to ethanol. An example of this type of proteins is the AdhE protein in E. coli (GenBank No: NP_415757). AdhE appears to be the evolutionary product of a gene fusion. The NH₂-terminal region of the AdhE protein is highly homologous to aldehyde:NAD⁺ oxidoreductases, whereas the COOH-terminal region is homologous to a family of Fe²⁺-dependent ethanol:NAD⁺ oxidoreductases (Membrillo-Hernández et al., (2000) J. Biol. Chem. 275: 33869-33875). The E. coli AdhE is subject to metal-catalyzed oxidation and therefore oxygen-sensitive (Tamarit et al. (1998) J. Biol. Chem. 273:3027-32).

2) Proteins that catalyze the reversible conversion of acetyl-CoA to acetaldehyde in strictly or facultative anaerobic micro-organisms but do not possess alcohol dehydrogenase activity. An example of this type of proteins has been reported in Clostridium kluyveri (Smith et al. (1980) Arch. Biochem. Biophys. 203: 663-675). An acetylating acetaldehyde dehydrogenase has been annotated in the genome of Clostridium kluyveri DSM 555 (GenBank No: EDK33116). A homologous protein AcdH is identified in the genome of Lactobacillus plantarum (GenBank No: NP_784141). Another example of this type of proteins is the aid gene product in Clostridium beijerinckii NRRL B593 (Toth et al. (1999) Appl. Environ. Microbiol. 65: 4973-4980. GenBank No: AAD31841).

3) Proteins that are involved in ethanolamine catabolismethanolamine can be utilized both as carbon and nitrogen source by many enterobacteria (Stojilkovic et al. (1995) J. Bacteriol. 177: 1357-1366). Ethanolamine is first converted by ethanolamine ammonia lyase to ammonia and acetaldehyde, subsequently, acetaldehyde is converted by acetylating acetaldehyde dehydrogenase to acetyl-CoA. An example of this type of acetylating acetaldehyde dehydrogenase is the EutE protein in Salmonella typhimurium (Stojilkovic et al. (1995) J. Bacteriol. 177: 1357-1366, GenBank No: AAL21357). E. coli is also able to utilize ethanolamine (Scarlett et al. (1976) J. Gen. Microbiol. 95:173-176) and has an EutE protein (GenBank No: AAG57564) which is homologous to the EutE protein in S. typhimurium.

4) Proteins that are part of a bifunctional aldolase-dehydrogenase complex a involved in 4-hydroxy-2-ketovalerate catabolism. Such bifunctional enzymes catalyze the final two steps of the meta-cleavage pathway for catechol, an intermediate in many bacterial species in the degradation of phenols, toluates, naphthalene, biphenyls and other aromatic compounds (Powlowski and Shingler (1994) Biodegradation 5, 219-236). 4-Hydroxy-2-ketovalerate is first converted by 4-hydroxy-2-ketovalerate aldolase to pyruvate and acetaldehyde, subsequently acetaldehyde is converted by acetylating acetaldehyde dehydrogenase to acetyl-CoA. An example of this type of acetylating acetaldehyde dehydrogenase is the DmpF protein in Pseudomonas sp CF600 (GenBank No: CAA43226) (Shingler et al. (1992) J. Bacteriol. 174:711-24). E. coli has a homologous MphF protein (FerrAndez et al. (1997) J. Bacteriol. 179: 2573-2581, GenBank No: NP_414885) to the DmpF protein in Pseudomonas sp. CF600.

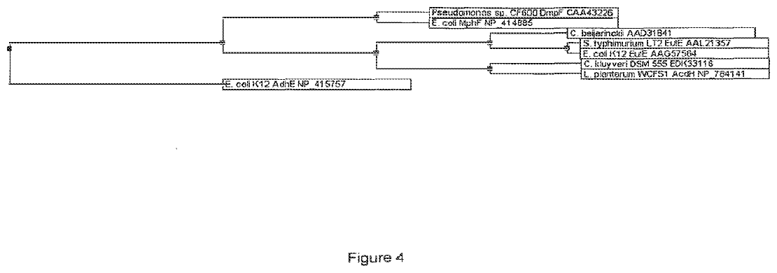

To identify the protein family members of acetylating acetaldehyde dehydrogenase, the amino acid sequences of the E. coli bifunctional AdhE protein (GenBank No: NP_415757), L. plantarum AcdH protein (acetylating) (GenBank No: NP_784141), the E. coli EutE protein (GenBank No: AAG57564) and the E. coli MhpF protein (GenBank No: NP_414885) were cach run as a query sequence in a BLASTp search against the GenBank non-redundant protein database using default parameters. Amino acid sequences with an E-value smaller or equal to 1e-20 were extracted. Redundant sequences were removed and the remaining sequences were aligned and a similarity tree was built using Genedata Physolopher protein analyzer software, version 6.5.2. A similarity tree provides information on organism sequence similarity. The tree is created independently of the ClustalW algorithm by pairwise comparison of the amino acid sequences per residue position. At each position, the similarity is rated and summed up to an overall score for each sequence pair. Based on these pairwise scores a hierarchical clustering is performed, which arranges the sequences in a tree. Note that the aid gene product of C. beijerinckii (GenBank no: AAD31841) clustered together with the EutE proteins from E. coli and S. typhimurium. From this similarity tree four major branches could be defined, each branch contains one amino acid sequence that was used as a query for the BLASTp search. FIG. 4 shows an example of such a similarity tree, containing all sequences that are mentioned in this example.

At least one amino acid sequence was selected from each branch for complementation tests in S. cerevisiae delta acs2. Preferably, the selected amino acid 3 sequences have experimental evidence of its biochemical function as acetylating acetaldehyde dehydrogenase. Such evidences can be found in public databases, such as in the BRENDA, UniProt and NCBI Entrez databases.

### Example 3

### Construction of Expression Plasmids and Complementation Test

To test whether acetylating acetaldehyde dehydrogenases (ACDH) could complement the deletion of ACS2 in S. cerevisiae, several genes coding for a (putative) ACDH were chosen from a variety of databases as described above.

To achieve optimal expression in yeast, the codon usage of all genes was adapted by codon pair optimization. These sequences were synthesized at Geneart AG (Regensburg, Germany).

The optimized sequences were cloned into the high copy yeast expression plasmid YEplac112PtdhTadh (SEQ ID NO:40; based on YEplac112 (2p TRP1) (Gietz & Sugino Gene 74(2):527-34), allowing constitutive expression from the TDH3 promoter.

YEplac112PtdhTadh was made by cloning a KpnI-SacI fragment from p426GPD (Mumberg et al. Gene, 156(1): 119-22), containing the TDH3 promoter and CYC1 terminator, into YEplac112 cut with KpnI-SacI. The resulting plasmid was cut with KpnI and SphI and the ends were made blunt then ligated to give YEplac112TDH. To obtain YEplac112PtdhTadh, YEplac112TDH was cut with PstI-HindIII and ligated to a 345 bp PstI-HindIII PCR fragment containing the ADH1 terminator (Tadh), thus replacing the CYC1 terminator and changing the polylinker between the promoter and terminator. The Tadh PCR fragment was generated using the following oligonucleotides:
MCS-5′Tadh: 5′-aaggtacctctagactagtcccgggctgcagtcgactcgagcga atttcttatgatttatgatt-3′
Tadh1-Hind: 5′-aggaagcttaggcctgtgtggaagaacgattacaacagg-3′

PCR was done with Vent^{R} DNA polymerase, according to the manufacturer's specifications.

The synthetic constructs containing the ACDH genes were cut with SpeI-PstI and ligated into YEplac112PtdhTadh digested with the same enzymes, resulting in pBOL58 through to pBOL68 and pBOL082. The names of the final plasmids and the genes they contain are given in Table 1.

TABLE 1
Overview on putative acetylating acetaldehyde dehydrogenases tested
for complementation of delta acs2 S. cerevisiae strain. Genes which
resulted in complementation are given in bold. SEQ ID NOs are
provided for the DNA sequence of the wild type gene, the protein
expressed therefrom, and the codon pair optimized DNA sequence.

| | | | | SEQ ID NO. |
|---|---|---|---|---|
| | | | Size | DNA/ |
| Organisms | Name | Group* | (kb) | PRT/OPT |
| Escherichia coli | adhE | 1 | 2.6 | |
| Entamoeba histolytica | adh2 | 1 | 2.6 | 48/50/49 |
| Staphylococcus aureus | adhE | 1 | 2.6 | 27/28/29 |
| Piromyces sp.E2 | adhE | 1 | 2.6 | 51/52 |
| Clostridium kluyveri | EDK33116 | 2 | 1.5 | 24/25/26 |
| Lactobacillus plantarum | acdH | 2 | 1.4 | |
| Escherichia coli | EutE | 3 | 1.4 | 18/19/20 |
| Listeria innocus | Lin1129 | 3 | 1.4 | 21/22/23 |
| Pseudomonas putida | YP 001268189 | 4 | 1.0 | |

*Group refers to the group of proteins having ACDH activity as defined in Example 2. Group 1: similar to bifunctional E. coli AdhE (AdhE-type of proteins); group 2; proteins having similarity to Lactobacillus plantarum AcdH (AcdH-type of proteins); group 3: similar to E. coli EutE (EutE-type of proteins); group 4: similar to E. coli MhpF (MhpF-type of proteins).

All plasmids were used to transform the delta acs2 yeast strain RWB060. As negative control, the empty vector YEplac112 was used. Transformants were plated on mineral medium (Verduyn et al. Yeast 8 (1992), pp. 501-517) containing either 1% glucose (MYD) or 1% ethanol+1% glycerol (MYEG) as single carbon source.

While for all constructs several transformants could be selected on minimal medium with ethanol/glycerol, this was not the case on the glucose containing plates.

TABLE 2
Result of a complementation experiment for putative acetylating
acetaldehyde dehydrogenases in delta acs2 S. cerevisiae strain
RWB060. Genes resulting in complementation are given in bold.
MYEG and MYD columns indicate number of transformants on plates
MYEG (ethanol/glycerol) and MYG (glucose).

| | Gene (GenPept | | | |
|---|---|---|---|---|
| Organisms | accession) | plasmid | MYEG | MYD |
| | none | YEplac112 | 75 | 0 |
| Escherichia coli | adhE | pBOL059 | 6 | 0 |
| Entamoeba histolytica | adh2 | pBOL061 | 54 | 0 |
| Staphylococcus aureus | adhE (BAB41363) | pBOL064 | 36 | 39 |
| Piromyces sp.E2 | adhE | pBOL139 | 32 | 3 |
| Clostridium kluyveri | EDK33116 | pBOL065 | 21 | 8 |
| | (EDK33116) | | | |
| Lactobacillus | acdH | pBOL058 | 6 | 0 |
| plantarum | | | | |
| Escherichia coli | EutE(ABV06849) | pBOL066 | 24 | 18 |
| Listeria innocus | Lin1129 | pBOL067 | 28 | 8 |
| | (CAC96360) | | | |
| Pseudomonas putida | YP 001268189 | pBOL068 | 32 | 0 |

On the glucose containing plates, transformants could only be selected for plasmids pBOL64, pBOL065, pBOL66, and pBOL67, not the empty vector. There was also a clear difference in colony size, depending on the plasmid used. While construct pBOL66 (E. coli eutE) resulted in biggest colonies, colonies of pBOL67 (L. innocua lin1129) appeared a bit smaller and pBOL065 (C. kluyveri edk3116) showed smallest colonies. Plasmid pBOLO64 (S. aureus adhE) and plasmid pBOL139 (Piromyces sp. E2, adhE) were done at a later date, so could not be compared directly, Colonies containing pBOL64 seemed to be similar to colonies comprising pBOL066 and colonies comprising pBOL139 seemed to be similar to colonies comprising pBOLO65.

To ensure that these results did not arise from spontaneous revertants, transformation experiments were repeated for some of the plasmids, giving the same results. In addition, for almost all plasmids four transformants were selected at random from the MYEG plates and restreaked onto MYD and MYEG plates.

In all experiments no growth was ever seen on glucose with the empty vector (YEplac112), while only pBOL065, pBOL066 and pBOL067 repeatedly gave good growth on glucose. Plasmid pBOL064 was not re-tested this way after the initial very positive result.

From these results, it was concluded that the codon pair optimized genes of the eutE homologues of:
E. coli (SEQ ID NO:20) encoding the ethanolamine utilization protein EutE from E. coli HS;
L. innocua (SEQ ID NO:23) encoding a hypothetical protein from L. innocua similar to ethanolamine utilization protein EutE, and
C. kluyveri (SEQ ID NO:26) encoding acetylating acetaldehyde dehydrogenase in Clostridium kluyveri DSM 555;
and the codon pair optimized gene of the adhE homologue of
S. aureus (SEQ ID NO-29) encoding a bifunctional acetaldehyde/alcohol dehydrogenase in Staphylococcus aureus subsp. aureus N315;
and the non codon pair optimized gene of the adhE homologue
Piromyces sp. E2 (SEQ ID NO:51) encoding a bifunctional acetaldehyde/alcohol dehydrogenase
are able to complement the acs2 yeast mutants. These genes encode an enzymatic activity allowing the formation of cytosolic acetyl-CoA from acetaldehyde in yeast.

### CONCLUSIONS

The supply of cytosolic acetyl-CoA is believed to be a bottleneck in the butanol production in yeast. In order to identify heterologous genes encoding for enzymes forming cytosolic acetyl-CoA in S. cerevisiae a test system based on a delta acs2 yeast mutant was established.

Due to its deficiency in cytosolic acetyl-CoA biosynthesis on glucose, the acs24 strain is unable to grow with glucose as sole carbon source.

9 putative acetylating acetaldehyde dehydrogenases identified as candidates for cytosolic acetyl-CoA supply from acetaldehyde were expressed in the acs24 yeast. In total, 5 of these 9 genes complemented growth of the acs24 strain with glucose as single carbon source. Therewith, the use of the delta acs2 strain as pre-selection tool for feasible routes for cytosolic supply of acetyl-CoA was shown.

4 of 5 acetylating acetaldehyde dehydrogenases identified thus far, eutE homologues of E. coli, L. innocua and C. kluyver and the adhE homologue of S. aureus, and Piromyces sp. E2, were successfully integrated in butanol producing strains of S. cerevisiae. The effect on butanol production was investigated as described in Examples below.

This test system may also be used, to analyse whether pyruvate:NADP oxidoreductase can successfully be over-expressed in yeast. Due to the oxygen sensitivity, this test has to be performed anaerobically.

Examples 4-6 below describe the testing 4 of the 5 selected ACDH genes from Example 3 for improvement of butanol production.

### Example 4

### Construction of a Butanol Producing Yeast Strain and Knocking Out the ADH1 and ADH2 Genes

The six Clostridium acetobutylicum genes involved in butanol biosynthesis from Acetyl-CoA are listed in Table 3. The genes were codon pair optimized for S. cerevisiae as described in WO2008/000632 and expressed from yeast promoters and terminators as listed in Table 3.

Two yeast integration vectors (pBOL34 [SEQ ID NO:41] and pBOL36 [SEQ ID NO:42]), each containing 3 of the six codon pair optimised genes from Clostridium acetobutylicum involved in butanol biosynthesis, were designed and synthesized at Geneart.

The genes ThiL, Hbd and Crt are expressed from pBOL34 containing a AmdS selection marker. The final three genes, Bcd, BdhB and AdhE were expressed from a integration vector with an AmdS selection marker named pBOL36.

TABLE 3
Genes used for butanol production in S. cerevisiae including
the promoter (1000 bp) and terminator (500 bp)

| Gene | activity | Promotor | Terminator |
|---|---|---|---|
| ThiL | acetyl CoA c-acetyltransferase | ADH1 | TDH1 |
| | [E.C. 2.3.1.9 | | |
| Hbd | 3-hydroxybutyryl-CoA | ENO1 | PMA1 |
| | dehydrogenase [E.C.1.1.1.157] | | |
| Crt | 3-hydroxybutyryl-CoA | TDH1 | ADH1 |
| | dehydrogenase [E.C.4.2.1.55] | | |
| Bcd | butyryl-CoA dehydrogenase | PDC1 | TDH1 |
| | [E.C.1.3.99.2]. | | |
| BdhB | NADH-dependent butanol | ENO1 | PMA1 |
| | dehydrogenase [E.C.1.1.1.−]. | | |
| adhE | alcohol/acetaldehyde CoA | TDH1 | ADH2 |
| | dehydrogenase [E.C.: 1.1.1.1/ | | |
| | 1.2.1.10] | | |

For integration in the ADH2 locus. pBOL36 was linearized by a BsaBI digestion. S. cerevisiae CEN.PK113-5D (MATa MAL2-8c SUC2 ura3-52) was transformed with the linear fragment and grown on plates with YCB (Difco) and 5 mM acetamide as nitrogen source.

The AmdS marker was removed by recombination by growing the transformants for 6 hours in YEPD in 2 ml tubes at 30° C. Cells were subsequently plated on 1.8% agar medium containing YCB (Difco) and 40 mM fluoroacetamide and 30 mM phosphate buffer pH 6.8 supporting growth only from cells that have lost the AmdS marker, Correct integration and recombination were confirmed by PCR. The correct integration of the fragment upstream was confirmed with the following primers:

P1:
5′-GAATTGAAGGATATCTACATCAAG-3′
and
P2:
5′-CCCATCTACGGAACCCTGATCAAGC-3′.

The correct integration of the fragment downstream was confirmed with the following primers:

P3:
5′-GATGGTGTCACCATTACCAGGTCTAG-3′
and
P4:
5′-GTTCTCTGGTCAAGTTGAAGTCCATTTTGATTGATTTGACTGTGTT
ATTTTGCGTG-3′.

The resulting strain was named BLT021.

pBOL34 was linearized by a Psil digestion and integrated in the ADH1 locus of BLT021. The transformants were grown on plates containing YCB (Difco) and 5 mM acetamide. For removal of the AmdS selection marker, colonies were inoculated in YEPD and grown for 6 hours in 2 ml tubes at 30° C. The cells were plated on YCB (Difco) and 40 mM fluoroacetamide and 0.1% ammonium sulphate.

Correct integration and recombination were confirmed by PCR. The correct integration of the fragment upstream was confirmed with the following primer set:

P5:
5′-GAACAATAGAGCGACCATGACCTTG-3′
and
P6:
5′-GACATCAGCGTCACCAGCCTTGATG-3′.

The correct integration of the fragment downstream was confirmed with the following primer set:

P7:
5′-GATTGAAGGTTTCAAGAACAGGTGATG-3′
and
P8:
5′-GGCGATCAGAGTTGAAAAAAAAATG-3′

The resulting strain was named BLT057.

### Example 5

### Introducing ETFα and ETFβ in BLT057

The ETF genes and the Acdh genes as listed in Table 4 were codon pair optimized for S. cerevisiae as described in WO2008/000632 and expressed from yeast promoters and terminators as listed in Table 4.

TABLE 4
Promoters and terminators used for expression of codon
pair optimized ETF-genes and Acdh genes in S. cerevisiae

| | Promotor | Terminator |
|---|---|---|
| Etfα(CpO) | tef1 | tdh2 |
| Etfβ(CpO) | tdh2 | tef1 |
| Acdh64 (AdhE S. aureus) | tdh3 | adh |
| Acdh65 (Clostridium) | tdh3 | adh |
| Acdh66 (EutE E. coli) | tdh3 | adh |
| Acdh67 (lin1129 Ec) | tdh3 | Adh |

The integration vectors expressing ETFα and ETFβ only (pBOL113, [SEQ ID NO:43]) or ETFα and ETFβ combined with Acdh64 (pBOL115, [SEQ ID NO:44]). Acdh65 (pBOL116, [SEQ ID NO:45]), Acdh66 (pBOL118, [SEQ ID NO:46]) or Acdh67 (pBOL120, [SEQ ID NO:47]) were synthesized by Geneart AG.

The vectors, pBOL113, pBOL115, pBOL116, pBOL118 and pBOL120, were linearized with StuI and integrated in the ura3-52 locus of strain BLT057.

The transformants were grown in YNB (Difco) w/o amino acids+2% galactose to select for uracil prototrophic strains. The strains derived from strain BLT057 with pBOL113/115/116/118/120 integrated in the genome were designated strains: BLT071, BLT072. BLT073. BLT074 and BLT075, respectively.

### Example 6

### Improved Butanol Production by Expressing Positive Acdh Genes

Strains BLT071 through BLT075 as prepared in Example 5 were grown in Verduyn medium (Verduyn et al. (1992) Yeast 8: 501-517) in which the ammonium sulphate is replaced by 2 g/l ureum and which further contains 4 wt. % galactose. Cells were grown in 100 ml shake flasks containing 50 ml of medium for 72 hours at 30° C. at 180 rpm in a rotary shaker.

The butanol concentration was determined in the supernatant of the culture. Samples were analysed on a HS-GC equipped with a flame ionisation detector and an automatic injection system. Column J & W DB-1 length 30 m, id 0.53 mm, df 5 μm. The following conditions were used: helium as carrier gas with a flow rate of 5 ml/min. Column temperature was set at 110° C. The injector was set at 140° C. and the detector performed at 300° C. The data was obtained using Chromeleon software. Samples were heated at 60° C. for 20 min in the headspace sampler. One (1) ml of the headspace volatiles were automatically injected on the column.

1-Butanol production of the various strains was as follows:
BLT057: 120 mg/l
BLT071: 450 mg/l
BLT072: 500 mg/l
BLT073: 600 mg/l
BLT074: 670 mg/l
BLT075: 700 mg/l

The results show that introduction of electron transfer flavoproteins (ETF alpha and ETF beta) and/or introduction of acetylating acetaldehyde dehydrogenases as identified by a complementation assay of Example 3, increase the butanol production level.

1. A method of identifying a heterologous polypeptide having enzymatic activity for converting pyruvate, acetaldehyde or acetate into acetyl-CoA in (the cytosol of) a yeast cell comprising:
providing a mutated yeast cell, wherein said mutation comprises an inactivation of at least one gene of the (PDH) by-pass, selected from the genes encoding the enzymes pyruvate decarboxylase (PDC), acetaldehyde dehydrogenase (ALD), and acetyl-CoA synthetase (ACS);
transforming said mutated yeast cell with an expression vector comprising at least one heterologous nucleotide sequence operably linked to a promoter functional in yeast and said heterologous nucleotide sequence encoding a candidate polypeptide having potential enzymatic activity for converting pyruvate, acetaldehyde or acetate into acetyl CoA;
testing said recombinant mutated yeast cell for its ability to grow on minimal medium containing glucose as sole carbon source, and
identifying said candidate polypeptide as a heterologous polypeptide having enzymatic activity for converting pyruvate, acetaldehyde or acetate into acetyl CoA in (the cytosol of) said yeast cell when growth of said cell is observed.

2. Method according to claim 1, wherein said yeast cell is a cell of Saccharomyces cerevisiae and wherein said heterologous nucleotide sequence is codon pair optimized for expression in Saccharomyces cerevisiae.

3. Method according to claim 2, wherein said mutation comprises an inactivation of the gene for acetyl-CoA synthetase isoform 2 (acs2).

4. Method according to claim 1, wherein said candidate polypeptide having enzymatic activity for converting acetaldehyde into acetyl-CoA is a (putative) acetylating acetaldehyde dehydrogenase (acdh).

5. A vector for the expression of heterologous polypeptides in yeast, said vector comprising a heterologous nucleotide sequence operably linked to a promoter functional in yeast and said heterologous nucleotide sequence encoding a polypeptide having enzymatic activity for converting pyruvate, acetaldehyde or acetate into acetyl-CoA in (the cytosol of) said yeast cell.

6. Vector according to claim 5, wherein said polypeptide having enzymatic activity for converting pyruvate, acetaldehyde or acetate into acetyl-CoA.

7. Vector according to claim 5, wherein said polypeptide has more than 50%, preferably more than 60%, 70%, 80%, 90%, or 95% sequence identity with the amino acid sequence selected from SEQ ID NO: 19, 22, 25, 28 and 52.

8. Vector according to claim 5, for expression in Saccharomyces cerevisiae, wherein said heterologous nucleotide sequence is codon pair optimized for expression in Saccharomyces cerevisiae.

9. Expression vector according to claim 8, wherein said heterologous nucleotide sequence is selected from SEQ ID NO: 20, 23, 26, 29 and 51.

10. A recombinant yeast cell comprising a vector of claim 5.

11. A recombinant yeast cell comprising a heterologous nucleotide sequence encoding a polypeptide having enzymatic activity for converting pyruvate, acetaldehyde or acetate into acetyl-CoA in (the cytosol of) said yeast cell.

12. Yeast cell according to claim 10, further comprising an inactivation of at least one gene of the (PDH) by-pass, selected from the genes encoding the enzymes pyruvate decarboxylase (PDC), acetaldehyde dehydrogenase (ALD), and acetyl-CoA synthetase (ACS).

13. Yeast cell according to claim 10, wherein the yeast cell comprises an inactivation of a gene encoding an acetyl-CoA synthase.

14. Yeast cell according to claim 10, wherein said cell shows growth on minimal medium containing glucose as sole carbon source.

15. Yeast cell according to claim 10, further comprising an inactivation of a gene encoding an enzyme that catalyses the conversion of acetaldehyde into ethanol, preferably an alcohol dehydrogenase.

16. Yeast cell according to claim 10, further comprising one or more introduced genes encoding a recombinant pathway for the formation of 1-butanol from acetyl-CoA.

17. Yeast cell according to claim 16, wherein said one or more introduced genes encode enzymes that produce acetoacetyl-CoA, 3-hydroxybutyryl-CoA, crotonyl-CoA, butyryl-CoA, butylaldehyde and/or 1-butanol.

18. Yeast cell according to claim 10, wherein said yeast is Saccharomyces cerevisiae.

19. A method of producing a fermentation product, comprising the steps of fermenting a suitable carbon substrate with a yeast cell according to claim 10 and recovering the fermentation product produced during said fermentation.

20. Method according to claim 19, wherein the fermentation product is butanol.


SEQUENCE LISTING (52 sequences)

SEQ ID NO: 1 - DNA, 64 bp, synthetic construct
  misc_feature 1..64: note = primer 5'acs2
  source 1..64: mol_type = other DNA
atacacaaacagaatacaggaaagtaaatcaatacaataataaaacagctgaagcttcgtacgc

SEQ ID NO: 2 - DNA, 67 bp, synthetic construct
  misc_feature 1..67: note = primer 3'acs2
  source 1..67: mol_type = other DNA
tctcattacgaaatttttctcatttaagttatttctttttttgaggcataggccactagtggatctg

SEQ ID NO: 3 - DNA, 20 bp, synthetic construct
  misc_feature 1..20: note = probe 5'acs2
  source 1..20: mol_type = other DNA
gatattcggtagccgattcc

SEQ ID NO: 4 - DNA, 20 bp, synthetic construct
  misc_feature 1..20: note = probe 3'acs2
  source 1..20: mol_type = other DNA
ccgtaaccttctcgtaatgc

SEQ ID NO: 5 - DNA, 20 bp, synthetic construct
  misc_feature 1..20: note = probe ACS2internal
  source 1..20: mol_type = other DNA
cggattcgtcatcagcttca

SEQ ID NO: 6 - DNA, 20 bp, synthetic construct
  misc_feature 1..20: note = probe KanA
  source 1..20: mol_type = other DNA
cgcacgtcaagactgtcaag

SEQ ID NO: 7 - DNA, 20 bp, synthetic construct
  misc_feature 1..20: note = probe KanB
  source 1..20: mol_type = other DNA
tcgtatgtgaatgctggtcg

SEQ ID NO: 8 - DNA, 64 bp, synthetic construct
  misc_feature 1..64: note = primer MCS-5'Tadh
  source 1..64: mol_type = other DNA
aaggtacctctagactagtcccgggctgcagtcgactcgagcgaatttcttatgatttatgatt

SEQ ID NO: 9 - DNA, 39 bp, synthetic construct
  misc_feature 1..39: note = primer Tadh1-Hind
  source 1..39: mol_type = other DNA
aggaagcttaggcctgtgtggaagaacgattacaacagg

SEQ ID NO: 10 - DNA, 24 bp, synthetic construct
  misc_feature 1..24: note = primer P1
  source 1..24: mol_type = other DNA
gaattgaaggatatctacatcaag

SEQ ID NO: 11 - DNA, 25 bp, synthetic construct
  misc_feature 1..25: note = primer P2
  source 1..25: mol_type = other DNA
cccatctacggaaccctgatcaagc

SEQ ID NO: 12 - DNA, 26 bp, synthetic construct
  misc_feature 1..26: note = primer P3
  source 1..26: mol_type = other DNA
gatggtgtcaccattaccaggtctag

SEQ ID NO: 13 - DNA, 56 bp, synthetic construct
  misc_feature 1..56: note = Primer P4
  source 1..56: mol_type = other DNA
gttctctggtcaagttgaagtccattttgattgatttgactgtgttattttgcgtg

SEQ ID NO: 14 - DNA, 25 bp, synthetic construct
  misc_feature 1..25: note = Primer P5
  source 1..25: mol_type = other DNA
gaacaatagagcgaccatgaccttg

SEQ ID NO: 15 - DNA, 25 bp, synthetic construct
  misc_feature 1..25: note = Primer P6
  source 1..25: mol_type = other DNA
gacatcagcgtcaccagccttgatg

SEQ ID NO: 16 - DNA, 27 bp, synthetic construct
  misc_feature 1..27: note = Primer P7
  source 1..27: mol_type = other DNA
gattgaaggtttcaagaacaggtgatg

SEQ ID NO: 17 - DNA, 25 bp, synthetic construct
  misc_feature 1..25: note = Primer P8
  source 1..25: mol_type = other DNA
ggcgatcagagttgaaaaaaaaatg

SEQ ID NO: 18 - DNA, 1404 bp, Escherichia coli
  source 1..1404: mol_type = other DNA
  CDS 1..1404
atgaatcaacaggatattgaacaggtggtgaaagcggtactgctgaaaatgcaaagcagtgacacgccgtccgccgccgttcatgagatgggcgttttcgcgtccctggatgacgccgttgcggcagccaaagtcgcccagcaagggttaaaaagcgtggcaatgcgccagttagccattgctgccattcgtgaagcaggcgaaaaacacgccagagatttagcggaacttgccgtcagtgaaaccggcatggggcgcgttgaagataaatttgcaaaaaacgtcgctcaggcgcgcggcacaccaggcgttgagtgcctctctccgcaagtgctgactggcgacaacggcctgaccctaattgaaaacgcaccctggggcgtggtggcttcggtgacgccttccactaacccggcggcaaccgtaattaacaacgccatcagcctgattgccgcgggcaacagcgtcatttttgccccgcatccggcggcgaaaaaagtctcccagcgggcgattacgctgctcaaccaggcgattgttgccgcaggtgggccggaaaacttactggttactgtggcaaatccggatatcgaaaccgcgcaacgcttgttcaagtttccgggtatcggcctgctggtggtaaccggcggcgaagcggtagtagaagcggcgcgtaaacacaccaataaacgtctgattgccgcaggcgctggcaacccgccggtagtggtggatgaaaccgccgacctcgcccgtgccgctcagtccatcgtcaaaggcgcttctttcgataacaacatcatttgtgccgacgaaaaggtactgattgttgttgatagcgtagccgatgaactgatgcgtctgatggaaggccagcacgcggtgaaactgaccgcagaacaggcgcagcagctgcaaccggtgttgctgaaaaatatcgacgagcgcggaaaaggcaccgtcagccgtgactgggttggtcgcgacgcaggcaaaatcgcggcggcaatcggccttaaagttccgcaagaaacgcgcctgctgtttgtggaaaccaccgcagaacatccgtttgccgtgactgaactgatgatgccggtgttgcccgtcgtgcgcgtcgccaacgtggcggatgccattgcgctagcggtgaaactggaaggcggttgccaccacacggcggcaatgcactcgcgcaacatcgaaaacatgaaccagatggcgaatgctattgataccagcattttcgttaagaacggaccgtgcattgccgggctggggctgggcggggaaggctggaccaccatgaccatcaccacgccaaccggtgaaggggtaaccagcgcgcgtacgtttgtccgtctgcgtcgctgtgtattagtcgatgcgtttcgcattgtttaa

SEQ ID NO: 19 - protein, 467 aa, Escherichia coli
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 20 - DNA, 1401 bp, synthetic construct
  misc_feature 1..1401: note = optimised sequence
  source 1..1401: mol_type = other DNA
atgaaccaacaagatatcgaacaagttgtcaaggctgtcttgttgaaaatgcaatcttctgacactccatctgctgctgtccacgaaatgggtgttttcgcttctttggacgacgctgttgctgctgccaaggttgctcaacaaggtttgaaatctgttgccatgagacaattggccattgctgccatcagagaagctggtgaaaagcatgccagagacttggctgaattggctgtctccgaaaccggtatgggtagagttgaagacaaattcgctaagaacgttgctcaagctagaggtactccaggtgtcgaatgtttgtctccacaagtcttgaccggtgataatggtttgactttgattgaaaatgctccatggggtgttgttgcttccgtcaccccatctaccaacccagctgctactgtcatcaacaacgccatctctttgattgctgctggtaactccgttatcttcgctccacacccagctgccaagaaggtttctcaaagagccatcactctattgaaccaagccattgttgctgctggtggtccagaaaacttgttggtcactgttgccaacccagatatcgaaactgctcaaagattattcaagttcccaggtatcggtctattagtcgtcactggtggtgaagctgttgttgaagctgccagaaagcacaccaacaagagattgattgctgctggtgctggtaaccctcctgttgttgtcgatgaaaccgctgatttggccagagctgctcaatccattgtcaagggtgcttctttcgacaacaacatcatctgtgctgacgaaaaggttttgattgttgttgactccgttgctgacgaattgatgagattgatggaaggtcaacatgccgtcaagttgactgctgaacaagctcaacaattgcaaccagttttgttgaagaacatcgatgaaagaggtaagggtaccgtctccagagactgggttggtagagatgctggtaagattgctgctgccatcggtttgaaggttccacaagaaaccagattattattcgtcgaaaccaccgctgaacacccatttgctgtcactgaattgatgatgccagtcttaccagttgtccgtgttgctaacgttgctgacgctattgctttggctgtcaaattggaaggtggttgtcaccacactgctgccatgcactccagaaacatcgaaaacatgaaccaaatggctaacgccattgacacttccatctttgtcaagaacggtccatgtatcgctggtttgggtttgggtggtgaaggttggaccaccatgaccatcaccaccccaactggtgaaggtgtcacttctgccagaactttcgtcagattacgtcgttgtgttttggtcgatgctttcagaattgtt

SEQ ID NO: 21 - DNA, 1410 bp, Listeria innocua
  source 1..1410: mol_type = other DNA
  CDS 1..1410
atggaatcattagaactcgaacaactggtaaaaaaagttctcttagaaaaattagcagaacaaaaagaagtaccaacaaaaacaactacacaaggcgcgaaaagtggcgtttttgatacagttgacgaggctgttcaagcagcagttatagcgcagaattgctataaagaaaaatcacttgaagaacgccgcaatgttgtaaaagcaattcgtgaagcactttatccagaaattgaaacaattgcgacaagagcagttgcagagactggtatgggaaatgtgacagataaaattttgaaaaacacgttagcaatcgaaaaaacgccaggggtagaagatttatatacagaagtagctacaggtgataacggtatgacactatatgaactctctccgtatggcgtaattggtgcagtagcgccgagcacaaacccaacggaaacattgatttgtaattcaatcggtatgctcgcagctggaaatgccgttttttatagccctcatccaggggcaaaaaacatttcactgtggttgattgaaaaactaaacacaattgttcgcgatagttgtggtatagataatctaattgtcaccgtggctaaaccatccatccaagcagctcaagaaatgatgaaccatccaaaagtaccgctacttgttattacaggtggtccgggcgttgttctccaagcgatgcaatcaggtaaaaaagtgattggagcaggagcagggaacccgccttctattgttgacgaaacagctaatatcgaaaaagcggctgctgacatcgtagacggagcatcttttgaccataatattttatgtattgctgaaaaaagtgtggtagctgttgatagcattgctgatttcttgttattccaaatggaaaaaaatggtgcccttcatgttactaatccaagtgatattcaaaaattagaaaaagtagccgttaccgataaaggtgtaactaataaaaaattagtcggaaaaagtgcaactgaaatcttaaaagaagcaggaatagcttgtgattttacaccacgtttaatcattgtggaaacggagaaatctcatccatttgcaacagtagagctattaatgccaatcgttccagttgtaagggtgcctgattttgacgaagcccttgaagtggctattgaactcgaacaaggcttacatcatacagcaacaatgcattcacaaaatatctcgagattaaacaaagctgcaagagatatgcaaacttccatctttgtcaaaaatggtccgtcctttgcgggattaggctttagaggagaaggtagtactactttcactattgcaacgcctactggagaaggaacaactacagcacgtcattttgctagacgccgccgctgtgttttaacagatggtttttcgattcgttaa

SEQ ID NO: 22 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 23 - DNA, 1407 bp, synthetic construct
  misc_feature 1..1407: note = optimised sequence
  source 1..1407: mol_type = other DNA
atggaatctttggaattggaacaattagtcaagaaggttttgttggaaaaattggctgaacaaaaggaagttccaaccaagaccaccacccaaggtgccaagtccggtgttttcgataccgtcgatgaagctgtccaagctgccgtcattgctcaaaactgttacaaggaaaaatctttggaagaaagaagaaacgttgtcaaggccatcagagaagctttatacccagaaatcgaaaccattgctaccagagctgttgctgaaaccggtatgggtaatgtcaccgataaaatcttgaagaacactttagctatcgaaaagactccaggtgttgaagacttgtacactgaagttgctaccggtgacaacggtatgactttatacgaattatctccatacggtgtcatcggtgctgttgctccatctaccaacccaactgaaactttgatctgtaactccatcggtatgttggctgctggtaacgccgttttctactctcctcacccaggtgccaagaacatctctttatggttgattgaaaagttgaacactatcgtcagagattcttgtggtattgacaacttgattgtcaccgttgccaagccatctatccaagctgctcaagaaatgatgaaccacccaaaggttccattgttggtcatcactggtggtccaggtgttgtcttgcaagctatgcaatctggtaagaaggttatcggtgctggtgctggtaaccctccatccatcgttgacgaaaccgctaacattgaaaaggctgctgctgacattgtcgacggtgcttcctttgaccataatatcttgtgtatcgctgaaaagtctgttgttgccgttgactccattgctgacttcttgttgttccaaatggaaaagaacggtgctttgcacgtcactaacccatctgatatccaaaaattggaaaaggttgccgtcactgacaagggtgtcaccaacaagaaattggttggtaagtctgccactgaaatcttgaaagaagctggtattgcttgtgatttcaccccaagattgatcattgtcgaaactgaaaagtcccacccattcgctactgttgaattgttgatgccaattgttccagttgtcagagttccagacttcgatgaagctttggaagttgccattgaattggaacaaggtctacatcacactgctaccatgcactctcaaaacatctccagattgaacaaggctgcccgtgacatgcaaacctccatctttgtcaagaacggtccatctttcgctggtttaggtttcagaggtgaaggttccaccactttcaccattgctactccaactggtgaaggtactaccactgcccgtcacttcgctagaagaagaagatgtgtcttgactgatggtttctccattaga

SEQ ID NO: 24 - DNA, 1476 bp, Clostridium kluyveri
  source 1..1476: mol_type = other DNA
  CDS 1..1476
atggagataatggataaggacttacagtcaatacaggaagtaagaactcttatagcaaaagcaaagaaagctcaagcagaatttaaaaatttttctcaagaagctgtaaacaaggtaatagaaaaaatagctaaggctacagaagttgaagctgtaaaacttgcaaaattggcatatgaagatacaggatatggaaaatgggaagataaagtaataaagaataagttttcaagtatagtagtttataactatattaaagatttgaaaacggttggaattttaaaagaagacaaggaaaagaaattaatagatatagctgttccacttggagttatagcaggacttataccttcaactaacccaacttcaacagcaatattcaaggtattaatagcattaaaggcaggaaatgcaatagtattctcaccacatccaacagcagtaagaagtattacagaaactgtaaagataatgcagaaagctgcagtagaagcaggagcaccagatggattaatccaatgtatgtcaatattgacagtagaaggtactgctgaattgatgaagaataaggatacagcacttatccttgcaacaggtggagaaggaatggtaagagcagcttacagttcaggaacaccagctataggagttggacctggaaacggcccatgctttattgaaagaacagcagatattcctacagcagtaagaaaagtaataggcagtgatacttttgataatggagtaatatgtgcttcagaacaatcaataatagcagagacagtaaagaaagcagagataattgaagaattcaagagacaaaaaggatatttcttaaatgcagaagaatcagaaaaagtaggcaagattttattaagagctaatggaacaccaaacccagcaatagtaggaaaagatgttcaagcattagcaaaattagcaggaataagcataccaagcgatgcggtaatattactttcagagcagacagatgtgagtccaaagaacccttatgcaaaggaaaaattagctccagtacttgcattctatacagtagaagactggcatgaagcatgtgaaaaatccttagcacttcttcataaccaaggaagtggacatacattaataattcactcacagaatgaagaaatcataagagaattcgcattgaagaaaccagtatcaagaatacttgtaaattcacctggatcacttggaggaataggtggagctacaaatcttgtaccatcacttacattaggctgtggagcagtaggtggaagtgcaacttcagataacgtaggaccagaaaacttattcaacataagaaaagtagcttatggaactacgacagtagaagaaataagagaagcttttggtgtaggagcagcttcatcaagtgcaccagcagaaccagaagataatgaagatgtacaggctatagtaaaagctataatggctaaattaaatctttaa

SEQ ID NO: 25 - protein, 491 aa, Clostridium kluyveri
MEIMDKDLQSIQEVRTLIAKAKKAQAEFKNFSQEAVNKVIEKIAKATEVEAVKLAKLAYEDTGYGKWEDKVIKNKFSSIVVYNYIKDLKTVGILKEDKEKKLIDIAVPLGVIAGLIPSTNPTSTAIFKVLIALKAGNAIVFSPHPTAVRSITETVKIMQKAAVEAGAPDGLIQCMSILTVEGTAELMKNKDTALILATGGEGMVRAAYSSGTPAIGVGPGNGPCFIERTADIPTAVRKVIGSDTFDNGVICASEQSIIAETVKKAEIIEEFKRQKGYFLNAEESEKVGKILLRANGTPNPAIVGKDVQALAKLAGISIPSDAVILLSEQTDVSPKNPYAKEKLAPVLAFYTVEDWHEACEKSLALLHNQGSGHTLIIHSQNEEIIREFALKKPVSRILVNSPGSLGGIGGATNLVPSLTLGCGAVGGSATSDNVGPENLFNIRKVAYGTTTVEEIREAFGVGAASSSAPAEPEDNEDVQAIVKAIMAKLNL

SEQ ID NO: 26 - DNA, 1473 bp, synthetic construct
  misc_feature 1..1473: note = optimised sequence
  source 1..1473: mol_type = other DNA
atggaaatcatggacaaggatttgcaatccatccaagaagttagaactttgattgccaaggccaagaaggctcaagctgaattcaagaacttttcccaagaagctgttaacaaggtcatcgaaaagatcgccaaggctactgaagttgaagctgtcaaattggccaaattggcttacgaagacaccggttacggtaaatgggaagacaaggtcatcaagaacaaattctcctccattgttgtctacaactacatcaaggatttgaagaccgttggtatcttgaaggaagacaaggaaaagaaattgattgacattgctgtcccattaggtgtcattgctggtttgattccatctaccaacccaacttccactgccattttcaaggtcttgattgctttgaaggctggtaacgccattgtcttctctccacacccaactgctgtccgttccatcactgaaaccgttaagatcatgcaaaaggctgctgttgaagctggtgctccagatggtttgatccaatgtatgtccattttgaccgttgaaggtactgctgaattgatgaagaacaaggacaccgctttgatcttggctaccggtggtgaaggtatggttagagctgcttactcctctggtactccagccatcggtgtcggtccaggtaacggtccatgtttcatcgaaagaactgctgacattccaactgctgttagaaaggttatcggttctgacactttcgacaacggtgtcatctgtgcttctgaacaatccatcattgctgaaaccgtcaagaaggctgaaatcatcgaagaattcaagagacaaaagggttacttcttgaatgctgaagaatctgaaaaggttggtaagattctattacgtgccaacggtactccaaacccagccatcgttggtaaggatgtccaagctttggccaaattggctggtatttccattccatctgatgctgttatcttactatccgaacaaaccgatgtttctcctaaaaatccatacgctaaggaaaaattggctccagtcttggctttctacaccgtcgaagactggcatgaagcttgtgaaaagtctttggctttattgcacaaccaaggttctggtcacactttgatcatccactctcaaaacgaagaaatcattagagaatttgctttgaagaagcctgtttccagaattttggttaactctccaggttctttgggtggtatcggtggtgctaccaacttagtcccatctttgactttaggttgtggtgctgttggtggttctgccacctctgacaacgttggtccagaaaacttgttcaacatcagaaaggttgcttacggtaccaccaccgtcgaagaaatcagagaagctttcggtgtcggtgctgcttcttcttctgctccagctgaaccagaagacaacgaagatgttcaagccattgttaaggccatcatggccaaattgaacttg

SEQ ID NO: 27 - DNA, 2610 bp, Staphylococcus aureus
  source 1..2610: mol_type = other DNA
  CDS 1..2610
atgttaactatacctgaaaaagaaaatcgtggatcgaaagaacaagaagtggcaattatgattgatgctctagctgacaaagggaaaaaagcattagaagcattatctaaaaagtcacaagaagaaattgatcatattgttcatcaaatgagcttagcagctgttgatcaacatatggtgctagcaaaattagcacatgaagaaactggaagaggtatatacgaagataaagcgattaaaaatttatacgcttctgaatatatatggaattcaataaaagacaataagacagtagggattattggtgaagataaagaaaaaggattaacgtatgtagcggaaccaattggtgttatttgtggtgttacgccaacaacaaatcctacgtcgacaactatttttaaagcgatgattgcaattaagacaggaaatccaatcatttttgcattccatccaagtgcacaagaatcgtcgaagcgtgcagcagaagttgtattagaagcggcaatgaaggcaggtgcacctaaagatattattcagtggattgaagtgccttctatcgaagcaacaaaacaattaatgaatcacaaaggtattgcattagttctagcaacaggtggttcgggcatggttaagtctgcatattcaactggcaaaccggcattaggtgtgggaccaggtaacgtgccgtcttacattgaaaaaacagcacacattaaacgtgcagtaaatgatatcattggttcaaaaacatttgataatggtatgatttgtgcttctgaacaagttgtagtcattgataaagaaatttataaagatgttactaatgaatttaaagcacatcaagcatactttgttaaaaaagatgaattacaacgcttagaaaatgcaattatgaatgaacaaaaaacaggtattaagcctgatattgtcggtaaatctgcagttgaaatagctgaattagcaggtatacctgtccccgaaaatacaaaacttatcatagccgaaattagcggtgtaggttcagactatccgttatctcgtgaaaaattatctccagtattagccttagtaaaagcccaatctacaaaacaagcatttcaaatttgtgaagacacactacattttggtggattaggacacacagccgttatccatacagaagatgaaacattacaaaaagattttggactaagaatgaaagcttgtcgtgtacttgtaaatacaccatcagcggttggaggtattggtgatatgtataacgaattgattccgtctttaacattaggttgtggttcgtacggtagaaactcaatttcacataatgttagtgcgacagatttattaaacattaaaacgattgctaaacgacgtaataatactcaaattttcaaggtgcctgctcaaatttattttgaagaaaatgcaatcatgagtctaacaacaatggacaagattgaaaaagtgatgattgtctgtgaccctggtatggtagaattcggttatacaaaaacagttgagaatgtattaagacaaagaacggaacagcctcaaattaaaatatttagcgaagtcgaaccgaacccatcaactaatacagtatataaaggtctggaaatgatggttgatttccaaccagatacaatcattgcacttggtggtggttcagcgatggatgctgcaaaagcaatgtggatgttctttgaacaccctgagacatcattcttcggtgctaaacaaaagttcctagacatcggtaaacgtacttataaaataggcatgcctgaaaatgcgacgttcatttgtatccctacgacatcaggtacaggttcagaagtaacaccatttgcagttatcacagatagtgaaacaaatgtaaaatatccgttggctgattttgctttaacacctgacgttgcaattattgaccctcaatttgtgatgagtgtgccaaaaagcgttacagcagatacaggaatggatgtactaacgcatgcaatggaatcatatgtatctgtaatggcttcagactatacaagaggtttgagtctacaagcgattaaattgacgttcgaatatttaaaatcatctgttgaaaagggtgataaagtttcaagagagaaaatgcataacgcatcaactttggctggtatggcatttgcaaatgcattcttaggcattgcacactcaattgcacataaaattggtggcgaatatggtattccgcatggtagagcgaatgcgatattactaccgcatattatccgttataatgccaaagacccgcaaaaacatgcattattccctaaatatgagttcttcagagcagatacagattatgcagatattgccaaattcttaggattaaaagggaatacgacagaagcactcgtagaatcattagctaaagctgtctacgaattaggtcaatcagtcggaattgaaatgaatttgaaatcacaaggtgtgtctgaagaagaattaaatgaatcaattgatagaatggcagagctcgcatttgaagatcaatgtacaactgctaatcctaaagaagcactaatcagtgaaatcaaagatatcattcaaacatcatatgattataagcaataa

SEQ ID NO: 28 - protein, 869 aa, Staphylococcus aureus
MLTIPEKENRGSKEQEVAIMIDALADKGKKALEALSKKSQEEIDHIVHQMSLAAVDQHMVLAKLAHEETGRGIYEDKAIKNLYASEYIWNSIKDNKTVGIIGEDKEKGLTYVAEPIGVICGVTPTTNPTSTTIFKAMIAIKTGNPIIFAFHPSAQESSKRAAEVVLEAAMKAGAPKDIIQWIEVPSIEATKQLMNHKGIALVLATGGSGMVKSAYSTGKPALGVGPGNVPSYIEKTAHIKRAVNDIIGSKTFDNGMICASEQVVVIDKEIYKDVTNEFKAHQAYFVKKDELQRLENAIMNEQKTGIKPDIVGKSAVEIAELAGIPVPENTKLIIAEISGVGSDYPLSREKLSPVLALVKAQSTKQAFQICEDTLHFGGLGHTAVIHTEDETLQKDFGLRMKACRVLVNTPSAVGGIGDMYNELIPSLTLGCGSYGRNSISHNVSATDLLNIKTIAKRRNNTQIFKVPAQIYFEENAIMSLTTMDKIEKVMIVCDPGMVEFGYTKTVENVLRQRTEQPQIKIFSEVEPNPSTNTVYKGLEMMVDFQPDTIIALGGGSAMDAAKAMWMFFEHPETSFFGAKQKFLDIGKRTYKIGMPENATFICIPTTSGTGSEVTPFAVITDSETNVKYPLADFALTPDVAIIDPQFVMSVPKSVTADTGMDVLTHAMESYVSVMASDYTRGLSLQAIKLTFEYLKSSVEKGDKVSREKMHNASTLAGMAFANAFLGIAHSIAHKIGGEYGIPHGRANAILLPHIIRYNAKDPQKHALFPKYEFFRADTDYADIAKFLGLKGNTTEALVESLAKAVYELGQSVGIEMNLKSQGVSEEELNESIDRMAELAFEDQCTTANPKEALISEIKDIIQTSYDYKQ

SEQ ID NO: 29 - DNA, 2607 bp, synthetic construct
  misc_feature 1..2607: note = optimised sequence
  source 1..2607: mol_type = other DNA
atgttgaccattccagaaaaggaaaacagaggttccaaggaacaagaagttgccatcatgattgatgctttagctgacaaaggtaagaaggctttggaagctttgtccaagaagtctcaagaagaaattgaccacattgtccaccaaatgtccttggctgctgttgaccaacacatggttttggccaagttggctcatgaagaaaccggtagaggtatctacgaagacaaggctatcaagaacttatacgcctctgaatacatctggaactccatcaaggacaacaagactgttggtatcattggtgaagacaaagaaaagggtttgacctacgttgctgaaccaattggtgtcatctgtggtgtcactccaaccaccaacccaacttctaccaccatcttcaaggctatgattgccatcaagactggtaacccaattattttcgctttccacccatctgctcaagaatcttccaagagagctgctgaagttgttttggaagctgccatgaaggctggtgctccaaaggatatcatccaatggattgaagttccatccattgaagctaccaagcaattgatgaaccacaagggtattgctttagtcttggctaccggtggttctggtatggttaagtctgcttactccactggtaaaccagctttgggtgttggtccaggtaacgttccatcttacatcgaaaagactgctcatatcaagcgtgctgtcaacgatatcatcggttccaagactttcgataatggtatgatctgtgcttctgaacaagttgttgtcattgacaaggaaatctacaaggatgtcaccaatgaattcaaggctcaccaagcttacttcgtcaagaaggacgaattacaaagattagaaaacgccatcatgaacgaacaaaagactggtatcaagccagatatcgttggtaagtctgctgttgaaattgctgaattggccggtatcccagttccagaaaacaccaaattgatcattgctgaaatctccggtgtcggttctgactacccattgtccagagaaaagttgtctccagttttggctttagtcaaggctcaatctaccaagcaagctttccaaatctgtgaagacactttgcacttcggtggtttaggtcacactgctgttatccacactgaagacgaaactttgcaaaaggatttcggtctaagaatgaaggcttgtcgtgttttggtcaacactccatctgctgttggtggtatcggtgacatgtacaacgaattgattccatccttgactttgggttgtggttcttacggtagaaactccatctcccacaacgtctctgctaccgatttgttgaacatcaagaccattgccaagagaagaaacaacactcaaatcttcaaggttccagctcaaatctatttcgaagaaaacgctatcatgtccttgaccaccatggacaagattgaaaaggtcatgatcgtttgtgacccaggtatggttgaatttggttacaccaaaaccgtcgaaaacgtcttacgtcaaagaactgaacaacctcaaatcaagatcttctctgaagttgaaccaaatccatccaccaacactgtctacaagggtttggaaatgatggtcgatttccaaccagacaccatcattgctttgggtggtggttctgccatggatgctgccaaggctatgtggatgttcttcgaacatccagaaacttctttcttcggtgccaagcaaaaattcttggacattggtaagagaacctacaagattggtatgccagaaaacgccactttcatctgtattccaaccacttctggtactggttctgaagtcactccatttgctgttatcactgactctgaaaccaacgtcaaatacccattggctgatttcgctttgactccagatgtcgccatcattgaccctcaatttgtcatgtccgtcccaaaatctgtcactgctgataccggtatggacgttttgactcacgctatggaatcttacgtttctgtcatggcctccgattacaccagaggtttgtccctacaagctatcaaattgacctttgaatacttgaaatcttccgttgaaaaaggtgacaaggtttccagagaaaagatgcacaacgcttctactttggccggtatggcctttgctaacgctttcttgggtattgctcactccattgctcacaaaattggtggtgaatacggtattccacatggtagagctaacgccatcttgttgcctcacatcatcagatacaacgccaaggaccctcaaaagcacgctttgttcccaaagtacgaattcttcagagctgacaccgattacgctgatatcgccaagttcttaggtttgaaaggtaacaccactgaagctttggttgaatctttggccaaggctgtctacgaattaggtcaatctgttggtattgaaatgaacttgaaatctcaaggtgtctctgaagaagaattgaacgaatccattgacagaatggctgaattggctttcgaagaccaatgtaccactgccaacccaaaggaagctttgatttctgaaatcaaggatatcatccaaacttcttacgactacaagcag

SEQ ID NO: 30 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 31 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 32 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 33 - protein, 379 aa, Clostridium acetobutylicum
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 34 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 35 - protein, 862 aa, Clostridium acetobutylicum
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 36 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 37 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 38 - protein, 336 aa, Clostridium acetobutylicum
MNKADYKGVWVFAEQRDGELQKVSLELLGKGKEMAEKLGVELTAVLLGHNTEKMSKDLLSHGADKVLAADNELLAHFSTDGYAKVICDLVNERKPEILFIGATFIGRDLGPRIAARLSTGLTADCTSLDIDVENRDLLATRPAFGGNLIATIVCSDHRPQMATVRPGVFEKLPVNDANVSDDKIEKVAIKLTASDIRTKVSKVVKLAKDIADIGEAKVLVAGGRGVGSKENFEKLEELASLLGGTIAASRAAIEKEWVDKDLQVGQTGKTVRPTLYIACGISGAIQHLAGMQDSDYIIAINKDVEAPIMKVADLAIVGDVNKVVPELIAQVKAANN

SEQ ID NO: 39 - protein, 259 aa, Clostridium acetobutylicum
MNIVVCLKQVPDTAEVRIDPVKGTLIREGVPSIINPDDKNALEEALVLKDNYGAHVTVISMGPPQAKNALVEALAMGADEAVLLTDRAFGGADTLATSHTIAAGIKKLKYDIVFAGRQAIDGDTAQVGPEIAEHLGIPQVTYVEKVEVDGDTLKIRKAWEDGYEVVEVKTPVLLTAIKELNVPRYMSVEKIFGAFDKEVKMWTADDIDVDKANLGLKGSPTKVKKSSTKEVKGQGEVIDKPVKEAAAYVVSKLKEEHYI

SEQ ID NO: 40 - DNA, 5976 bp, synthetic construct
  misc_feature 1..5976: note = plasmid YEplac112PtdhTadh
  source 1..5976: mol_type = other DNA
gcccgggggatccactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatcggatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcgatccgatgataagctgtcaaacatgagaattgatcttttatgcttgcttttcaaaaggcttgcaggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctctcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagtgcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaggcctgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgctcgagtcgactgca

SEQ ID NO: 41 - DNA, 13286 bp, synthetic construct
  misc_feature 1..13286: note = pBOL34
  source 1..13286: mol_type = other DNA
aagcttgcatgcctgcaggtcgacggcgcgccgggcccgtttaaacggccggccaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttccgatttttttctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatatacaatgaaggaagttgttattgcttctgctgtcagaactgccattggttcttacggtaagtctttgaaggacgtcccagctgtcgacttgggtgctaccgccatcaaggaagctgtcaagaaggctggtatcaagccagaagatgttaacgaagttatcttaggtaacgttttgcaagctggtttaggtcaaaacccagctcgtcaagcttctttcaaggctggtttgccagttgaaattccagccatgaccatcaacaaggtttgtggttctggtttgagaactgtttctttggctgctcaaatcatcaaggctggtgacgctgatgtcatcattgctggtggtatggaaaacatgtccagagctccatacttggctaacaatgctagatggggttacagaatgggtaacgccaagttcgtcgatgaaatgatcactgacggtttatgggacgctttcaacgactaccacatgggtatcactgctgaaaacattgctgaaagatggaacatctccagagaagaacaagatgaatttgctttggcttctcaaaagaaggctgaagaagccatcaaatctggtcaattcaaggacgaaattgtcccagttgtcatcaagggtagaaagggtgaaaccgttgtcgacaccgatgaacacccaagattcggttccaccattgaaggtttggccaagttgaaaccagctttcaagaaggatggtaccgtcactgctggtaacgcttccggtttgaacgactgtgctgctgttttggttatcatgtctgctgaaaaggccaaggaattgggtgtcaagccattggccaagattgtctcctacggttctgctggtgttgacccagccatcatgggttacggtcctttctacgctaccaaggctgctatcgaaaaggctggttggaccgttgacgaattggatttgattgaatccaacgaagctttcgctgctcaatctttggctgttgccaaggacttgaaattcgacatgaacaaggtcaacgttaacggtggtgccattgctttgggtcacccaattggtgcttccggtgccagaatcttggttactttagtccacgctatgcaaaagcgtgatgccaagaagggtttggctactctatgtatcggtggtggtcaaggtactgccatcttattggaaaagtgttaggcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaagtttaaacatttaaacgtgtgtgtgcattatatatattaaaaattaagaattagactaaataaagtgtttctaaaaaaatattaaagttgaaatgtgcgtgttgtgaattgtgctctattagaataattatgacttgtgtgcgtttcatattttaaaataggaaataaccaagaaagaaaaagtaccatccagagaaaccaattatatcaaatcaaataaaacaaccagcttcggtgtgtgtgtgtgtgtgaagctaagagttgatgccatttaatctaaaaattttaaggtgtgtgtgtggataaaatattagaatgacaattcgagatgaaattttaagcaaactctagtaggaaataagcggcttattcttgttggctcctaattctttttagtgtatcagttcccattgataaaaaaattaaaattaaaattagaaaaattaaaccagaaaaatcaagttgattaaaatgtgacaaaaattatgattaaatgctacttcaacaggagcccgggcctatttggagtagtcgtagaaacccttaccagactttctacctaaccaaccagctctaacgtacttcttcaataaagtgtgaggtctgtacttagagtcaccggtttcagagtataagacatccatgatggccaaacagatatccaaaccgatgaagtcacctaattccaatggacccattgggtggttagcacccaatttcatggccttgtcgatatcttcaacagaagcaataccttcagccaaaataccgacagcttcgttgatcattggaatcaagattctgttgacaacgaaacctggagcttcagcaacttcaactgggtccttaccaatggcaatggaagtttccttgacagcatcgaaagtttcttgagaggtggcaatacctctgatgacttcgaccaacttcatgactggagctgggttgaagaagtgcataccgataaccttgtctggtctcttggtagcagaagcaacttcagtgatggacaaagaagaagtgttggaagccaaaatggtttctggcttacagatgttgtccaaatcagcaaagatttgcttcttgatgtccattctttcaacggcagcttcaatgaccaaatcacagtcagcagccatgttcaagtcaacagtaccggagattctggtcaagatttcgaccttggtagcttcttcaatcttacccttcttgaccaacttggacaagttcttgttgatgaaatccaaaccacggtcaacgaattcgtccttgatatctctcaaaacaacttcgaaacccttggcagcgaaagcttgagcaataccagaacccatggtaccggcaccaatgacacaaaccttcttcattttgatttagtgtttgtgtgttgataagcagttgcttggttttttatgaaaaatagctagaaggaataagggattacaagagagatgttacaagaaagaagtaaaataaatttgattaatattgccattatcaaaagctatttatatgttgaaatcgtggagatcatgtgtgccagaaaaggccacagtttccggggagaggcataccttgaggtggctaggaatcacggagacctcttgacttgcagggtaggctagctagaattaagtgaggtgacaaggtttccatacagttttgaccttgagacgttgctacttacgatttgcagtatgcaagtctcatgctgcaaacaaaagaggaccgctcaggtaatcgctcaattagtggacgttatcaggggcgggagaggcgaaagtggtttttggtggtgtaagtaaaggtcgtccaaatatgcaggtgtttgggtgctatcctagtggaagctcggatcagtagataacccgcctagaagcggtatttttctttttttttcttccttctttttcgtcattatttcaaacgcttttgcgtcaagtaatgaatatctggcggttccgcggtaatgcgacaatttgtgatatgcactcttaaaaccccgccacgatgatcgcacgtgccggcatttatagacgacttttctggttgtcccgcttcacggcacatgcatgcatcaatgaccgaattcaggttgctactaaccattgtgttgtgttattgctgtgcatgaggtgctcaagtgcccgcggcatctgactagtggtaactctagacggcttcgatgcagagagttcctcaaaatttttcttttcaattgtttgcctggtttccgcggcgtatatcagtttttggcgatatggtaacgcgatactctacggcaccttcacggtagatgtcttttttaaaagtgactgttaattccaggattgaaaggaagtgtcgaatagtatagtatgctttctaggccggccgtttaaatgggcccgcggcccgtttaaacggccggcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacataacaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatggaattgaacaacgttatcttggaaaaggaaggtaaggttgccgttgtcaccatcaacagaccaaaggctttgaatgctttgaactctgacactttgaaggaaatggactacgtcattggtgaaattgaaaacgattctgaagttttggctgtcatcttgaccggtgccggtgaaaagtctttcgttgctggtgctgatatctctgaaatgaaggaaatgaacaccattgaaggtagaaagttcggtatcttaggtaacaaggttttcagaagattggaattgttggaaaagccagtcattgctgctgtcaacggtttcgctttgggtggtggttgtgaaattgccatgtcctgtgacatcagaattgcttcttctaacgctcgtttcggtcaaccagaagtcggtctaggtatcactccaggtttcggtggtactcaaagattatccagattggttggtatgggtatggccaagcaattgatcttcaccgctcaaaacatcaaggctgacgaagctttgagaattggtttagtcaacaaggttgttgaaccatctgaattgatgaacactgccaaggaaattgctaacaagatcgtctccaacgctccagttgctgtcaaattgtccaagcaagccatcaacagaggtatgcaatgtgatatcgacaccgctttggcctttgaatctgaagctttcggtgaatgtttctccactgaagaccaaaaggatgctatgaccgctttcatcgaaaagagaaagattgaaggtttcaagaacaggtgatgagcccgggcgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacggatccacagcctagccttcagttgggctctatcttcatcgtcattcattgcatctactagccccttacctgagcttcaagacgttatatcgcttttatgtatcatgatcttatcttgagatatgaatacataaatatatttactcaagtgtatacgtgcatgctttttttacggtttaaacatttaaatgggccgctctagaggatccccgggtaccgagctcgggcccagcgctactagttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagagacccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtcgagaatttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatcaaagtaaaaaaaagattttcgctacatgttccttttgaagaaagaaaatcatggaacattagatttacaaaaatttaaccaccgctgattaacgattagaccgttaagcgcacaacaggttattagtacagagaaagcattctgtggtgttgccccggactttcttttgcgacataggtaaatcgaataccatcatactatcttttccaatgactccctaaagaaagactcttcttcgatgttgtatacgttggagcatagggcaagaattgtggcttgagatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 42 - DNA, 16359 bp, synthetic construct
  misc_feature 1..16359: note = pBOL36
  source 1..16359: mol_type = other DNA
aagcttgcatgcctgcaggtcgacggcgcgccgggcccgtttaaacaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtgggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacaggccggccaaaatgaaggccaaatcaaggcgggaagggacaaccaggacgtaaagggtagcctccccataacataaactcaataaaatatatagtcttcaacttgaaaaaggaacaagctcatgcaaagaggtggtacccgcacgccgaaatgcatgcaagtaacctattcaaagtaatatctcatacatgtttcatgagggtaacaacatgcgactgggtgagcatatgttccgctgatgtgatgtgcaagataaacaagcaagacagaaactaacttcttcttcatgtaataaacacaccccgcgtttatttacctatctttaaacttcaacaccttatatcataactaatatttcttgagataagcacactgcacccataccttccttaaaaacgtagcttccagtttttggtggttctggcttccttcccgattccgcccgctaaacgcataattttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccgtttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttggaatcagatttgggtttgttccctttattttcatatttcttgtcatattcttttctcaattattatcttctactcataacctcacgcaaaataacacagtcaaatcaatcaaaatggacttcaacttgaccagagaacaagaattggtcagacaaatggttagagaatttgctgaaaacgaagttaagccaattgctgctgaaatcgatgaaactgaaagattcccaatggaaaacgtcaagaagatgggtcaatacggtatgatgggtattccattctctaaggaatacggtggtgctggtggtgacgtcttgtcttacatcattgctgtcgaagaattgtccaaggtttgtggtaccactggtgtcatcttatctgctcacacttctctatgtgcctccttgatcaacgaacacggtactgaagaacaaaagcaaaagtacttggttccattggccaagggtgaaaagattggtgcctacggtttgactgaaccaaacgctggtactgactctggtgctcaacaaactgttgccgttttggaaggtgaccactacgtcatcaacggttccaagatcttcatcaccaacggtggtgttgctgacacctttgtcatcttcgctatgaccgatcgtaccaagggtaccaagggtatctctgctttcattattgaaaagggtttcaagggtttctccatcggtaaggtcgaacaaaagttgggtatcagagcttcctctaccactgaattggttttcgaagacatgattgttccagttgaaaacatgatcggtaaggaaggtaagggtttcccaattgccatgaagactttagatggtggtagaattggtattgctgctcaagctttgggtattgctgaaggtgccttcaacgaagctagagcttacatgaaggaaagaaagcaattcggtagatctttggacaaattccaaggtttggcttggatgatggctgacatggacgttgccatcgaatctgctcgttacttggtctacaaggctgcttacttgaagcaagctggtttgccatacaccgtcgatgctgccagagctaagttgcacgctgccaacgttgccatggatgtcaccaccaaggctgtccaattattcggtggttacggttacaccaaggactacccagttgaaagaatgatgagagatgctaagatcactgaaatctacgaaggtacttctgaagttcaaaagttggttatctccggtaagatcttcagataggcccgggcataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgcctttgcaaaacgcacatacccacaatatgtgactattggcaaagaacgcattatcctttgaagaggtggatactgatactaagagagtctctattccggctccacttttagtccagagattacttgtcttcttacgtatcagaacaagaaagcatttccaaagtaattgcatttgcccttgagcagtatatatatactaagaagtttaaacatttaaacggccggcctagaaagcatactatactattcgacacttcctttcaatcctggaattaacagtcacttttaaaaaagacatctaccgtgaaggtgccgtagagtatcgcgttaccatatcgccaaaaactgatatacgccgcggaaaccaggcaaacaattgaaaagaaaaattttgaggaactctctgcatcgaagccgtctagagttaccactagtcagatgccgcgggcacttgagcacctcatgcacagcaataacacaacacaatggttagtagcaacctgaattcggtcattgatgcatgcatgtgccgtgaagcgggacaaccagaaaagtcgtctataaatgccggcacgtgcgatcatcgtggcggggttttaagagtgcatatcacaaattgtcgcattaccgcggaaccgccagatattcattacttgacgcaaaagcgtttgaaataatgacgaaaaagaaggaagaaaaaaaaagaaaaataccgcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaaatggtcgatttcgaatactctatcccaaccagaatcttcttcggtaaggacaagatcaacgttttgggtagagaattgaagaaatacggttccaaggttttgattgtctacggtggtggttccatcaagagaaacggtatctacgacaaggctgtctccattttggaaaagaactctatcaaattctacgaattggctggtgttgaaccaaacccaagagttaccaccgtcgaaaagggtgtcaagatctgtcgtgaaaacggtgttgaagttgttttggccatcggtggtggttctgccattgactgtgccaaggtcattgctgctgcctgtgaatacgatggtaacccatgggacattgtcttggatggttctaagatcaagcgtgtcttaccaattgcttccatcttgactatcgctgctactggttctgaaatggacacctgggctgttatcaacaacatggacactaacgaaaagttgattgctgctcacccagatatggccccaaagttctctattttggacccaacctacacttacactgttccaaccaaccaaactgctgctggtactgctgatatcatgtctcacatctttgaagtttacttctccaacaccaagaccgcttacttgcaagacagaatggctgaagctctattaagaacctgtatcaagtacggtggtattgctttggaaaagccagatgactacgaagccagagctaacttgatgtgggcttcctctttggctatcaacggtttattgacttacggtaaggacaccaactggtccgttcatttgatggaacacgaattgtctgcttactacgatatcactcacggtgtcggtttggccatcttgactccaaactggatggaatacattttgaacaacgacactgtctacaagttcgtcgaatacggtgttaacgtctggggtattgacaaggaaaagaaccactacgacattgctcaccaagccatccaaaagaccagagactatttcgtcaacgttttgggtttaccatccagattaagagatgttggtattgaagaagaaaaattggatatcatggctaaggaatctgtcaaattgactggtggtaccattggtaacttgagacctgttaacgcttctgaagttttgcaaatcttcaagaaatctgtttaggcccgggctcctgttgaagtagcatttaatcataatttttgtcacattttaatcaacttgatttttctggtttaatttttctaattttaattttaatttttttatcaatgggaactgatacactaaaaagaattaggagccaacaagaataagccgcttatttcctactagagtttgcttaaaatttcatctcgaattgtcattctaatattttatccacacacacaccttaaaatttttagattaaatggcatcaactcttagcttcacacacacacacacaccgaagctggttgttttatttgatttgatataattggtttctctggatggtactttttctttcttggttatttcctattttaaaatatgaaacgcacacaagtcataattattctaatagagcacaattcacaacacgcacatttcaactttaatatttttttagaaacactttatttagtctaattcttaatttttaatatatataatgcacacacacgtttaaatgggcccgcggcccgtttaaacggccggcccttcccttttacagtgcttcggaaaagcacagcgttgtccaagggaacaatttttcttcaagttaatgcataagaaatatctttttttatgtttagctaagtaaaagcagcttggagtaaaaaaaaaaatgagtaaatttctcgatggattagtttctcacaggtaacatagcaaaaaccaagaaaagcccgcttctgaaaactacagttgacttgtatgctaaagggccagactaatgggaggagaaaaagaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttgcgctaggaataggatatgcgacgaagacgcttctgcttagtaaccacaccacattttcagggggtcgatctgcttgcttcctttactgtcacgagcggcccataatcgcgctttttttttaaaaggcgcgagacagcaaacaggaagctcgggtttcaaccttcggagtggtcgcagatctggagactggatctttacaatacagtaaggcaagccaccatctgcttcttaggtgcatgcgacggtatccacgtgcagaacaacatagtctgaagaagggggggaggagcatgttcattctctgtagcagtaagagcttggtgataatgaccaaaactggagtctcgaaatcatataaatagacaatatattttcacacaatgagatttgtagtacagttctattctctctcttgcataaataagaaattcatcaagaacttggtttgatatttcaccaacacacacaaaaaacagtacttcactaaatttacacacaaaacaaaatgaaggttaccaaccaaaaggaattgaagcaaaagttgaacgaattgagagaagctcaaaagaagttcgctacctacactcaagaacaagttgacaagatcttcaagcaatgtgccattgctgctgccaaggaacgtatcaacttggccaagttggctgtcgaagaaaccggtattggtttggttgaagacaagatcatcaagaaccacttcgctgctgaatacatctacaacaagtacaagaacgaaaagacctgtggtatcatcgaccacgatgactctttgggtatcaccaaggttgctgaaccaatcggtattgtcgccgccattgtcccaaccactaacccaacttccactgccatcttcaaatctttgatctccttgaagaccagaaacgctatcttcttctccccacacccaagagccaagaagtccaccattgctgctgccaaattaatcttggatgctgctgttaaggctggtgccccaaagaacattattggttggatcgatgaaccttccattgaattgtctcaagacttgatgtctgaagctgatatcatcttggctaccggtggtccatccatggtcaaggccgcttactcttctggtaagccagctattggtgttggtgctggtaacactccagctatcatcgatgaatctgctgacattgacatggctgtctcctccattatcttgtccaagacttatgacaacggtgtcatctgtgcctctgaacaatccatcttggttatgaactctatctacgaaaaggtcaaggaagaatttgttaagagaggttcctacatcttaaaccaaaatgaaattgccaagatcaaggaaaccatgttcaagaacggtgccatcaacgctgacattgtcggtaaatctgcttacatcattgccaagatggctggtattgaagttccacaaaccactaagattttgatcggtgaagttcaatctgtcgaaaagtctgaattattctctcacgaaaagttgtctccagtcttggctatgtacaaggtcaaggatttcgacgaagctttgaagaaggctcaaagattaattgaattaggtggttctggtcacacctcttctctatacattgactctcaaaacaacaaggacaaggtcaaggaattcggtctagctatgaagacttccagaactttcatcaacatgccatcttctcaaggtgcttctggtgatttgtacaactttgccattgctccatctttcactttaggttgtggtacctggggtggtaactctgtttctcaaaacgttgaaccaaagcatttgctaaacatcaagtccgttgctgaaagaagagaaaacatgttgtggttcaaggttccacaaaagatctacttcaaatacggttgtttgagatttgctttgaaggaattgaaagatatgaacaagaagcgtgctttcatcgttactgacaaggatttgttcaaattgggttacgttaacaagatcactaaggttttggatgaaattgatatcaagtactccatcttcactgatatcaaatctgacccaaccattgactccgtcaagaagggtgctaaggaaatgttgaacttcgaaccagataccattatctccattggtggtggttctccaatggatgctgccaaggttatgcatttgttgtacgaatacccagaagctgaaatcgaaaacttggccatcaacttcatggacatcagaaagagaatctgtaacttcccaaagttgggtaccaaggccatttctgttgccattccaaccaccgctggtaccggttctgaagctactccatttgctgtcatcaccaacgacgaaaccggtatgaagtacccattgacctcttacgaattgactccaaacatggccatcattgacactgaattgatgttgaacatgccaagaaagttgactgctgctaccggtattgacgctttagtccacgctatcgaagcttacgtctccgttatggccactgactacactgacgaattggctttgagagctatcaagatgatcttcaagtacttgccaagagcttacaagaacggtactaacgatatcgaagctcgtgaaaagatggctcacgcttccaacattgctggtatggctttcgctaacgctttcttgggtgtttgtcactccatggcccacaagttgggtgctatgcaccacgttcctcacggtattgcttgtgctgttttgattgaagaagtcatcaagtacaacgctactgactgtccaaccaagcaaactgctttcccacaatacaagtctccaaacgccaagagaaagtacgctgaaattgctgaatacttgaacttgaaaggtacttctgacactgaaaaggtcactgctttaatcgaagctatctccaagttgaagattgacttatctattcctcaaaacatctctgctgctggtattaacaagaaggacttctacaacactttagacaagatgtccgaattggctttcgatgaccaatgtaccaccgctaacccaagatacccattgatctctgaattgaaggatatctacatcaagtccttttaagcccgggcgcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccagcattgaaatgatcgaagttcaatgatgaaacggtaattcttctgtcatttactcatctcatctcatcaagttatataattctatacggatgtaatttttcacttttcgtcttgacgtccaccctataatttcaattattgaaccctcacaaatgatgcactgcaatgtacacaccctcatatagtttaaacatttaaatgggccgctctagaggatccccgggtaccgagctcgggcccagcgctactagttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgagacctctgacatgcttttgacaggaatagacattttcagaatgttatccatatgcctttcgggtttttttccttccttttccatcatgaaaaatctctcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagctagttaaagaaaatcattgaaaagaataagaagataagaaagatttaattatcaaacaatatcaatatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttaacaaactgcgcccacgagttcttccctgacgccgctctcgcgcaggcaagggaactcgatgaatactacgcaaagcacaagagacccgttggtccactccatggcctccccatctctctcaaagaccagcttcgagtcaagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgaggacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtcgagaatttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggattaattacttggatgccaataaaaaaaaaaaacatcgagaaaatttcagcatgctcagaaacaattgcagtgtatcaaagtaaaaaaaagattttcgctacatgttccttttgaagaaagaaaatcatggaacattagatttacaaaaatttaaccaccgctgattaacgattagaccgttaagcgcacaacaggttattagtacagagaaagcattctgtggtgttgccccggactttcttttgcgacataggtaaatcgaataccatcatactatcttttccaatgactccctaaagaaagactcttcttcgatgttgtatacgttggagcatagggcaagaattgtggcttgagatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 43 - DNA, 8684 bp, synthetic construct
  misc_feature 1..8684: note = pBOL113
  source 1..8684: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccatttaaacggccggccctagatcagagggtggtaaatgaagtgtaatagtattcatttttcttataaatcatcccttccgtgatttatacaaaagaagaggagaatatgctgaatacttggtatattactctacattatactcttatcttgacgggtattctgagcatcttactcagtttcaagatcttttaatgtccaaaaacatttgagccgatctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaaatgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaaagcccgggcggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgtttaaatgtttaaacatggacagatatgcgatgaaaacgctaagtgatactccaaatggtgaaaggtacgatgcttggaaacaatacttggaaatcaccggaaacaccatatgcggcgaaaagccaattagtgtgatactaagtgctttatcgaaaatccgtgatgccggtccttcaggcatcaaatttcagtggcctaattattcacagagttctcatgtgacaagtattgatgatagtagtgtcagttatgcttcaggttatgttactataggataatgatcacggctaaaacggtcgaatgtaagcatatatctttcgattgtataattgttcccaaatactacagcatctcaaggaaaaaaaaacaaaaacttccaaaaaaatcgaatccctgaggaatctttaatacattttcaatctatttaagttttataaacgtgtatatgagatgtcatgagcatgaattattaataataaaaactaaatcattaaagtaacttaaggagttaaagcccgggctttaattgttagcagccttgacttgagcaatcaattctggaacaaccttgttgacatcaccgacaatggccaaatcagcaaccttcatgattggagcttcgacatctttgttgatggcaatgatgtagtcagagtcttgcataccagccaagtgttggatggcaccagagataccacaagcaatgtacaaagttggtctgacggtcttaccggtttgaccgacttgcaagtccttgtcaacccattccttttcaatggcagctctggaagcagcaatggtaccacccaacaaagaagctaattcttccaatttttcgaagttttccttggaaccaacaccacgaccaccagcaaccaaaaccttggcttcaccgatatcagcaatgtccttggccaatttgacaaccttggaaaccttggttctgatatcagaagcagtcaatttgatggcaaccttttcgatcttgtcatcagaaacgttagcatcgttaactggcaatttttcaaagacacctggtctgacggtggccatttgaggtctgtggtcagaacagacaatggtagcaatcaagttaccaccgaaagctggtctggtagccaacaagtcacggttttcgacatcgatatccaaagaggtacagtcagcagtcaaaccagtagacaatctggcagcaattcttggacccaagtctctaccgatgaaagtagcaccgatgaataagatttctggctttctttcgttgaccaagtcacagataaccttggcgtaaccgtcagtggagaaatgagctaataattcgttgtcagcagccaaaaccttgtcagcaccgtgggacaacaagtccttggacatcttttcagtgttgtgacccaataagacagcagtcaattcaacacccaatttttcagccatttccttacccttacctagcaattccaaagaaaccttttgtaattcaccatctctttgttcagcgaaaacccagacacccttgtagtcagccttgttcatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaggccggccgtttaaacgggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 44 - DNA, 12314 bp, synthetic construct
  misc_feature 1..12314: note = pBOL115
  source 1..12314: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccatttaaacggccggccctagatcagagggtggtaaatgaagtgtaatagtattcatttttcttataaatcatcccttccgtgatttatacaaaagaagaggagaatatgctgaatacttggtatattactctacattatactcttatcttgacgggtattctgagcatcttactcagtttcaagatcttttaatgtccaaaaacatttgagccgatctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaaatgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaaagcccgggcggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgtttaaatgtttaaacatggacagatatgcgatgaaaacgctaagtgatactccaaatggtgaaaggtacgatgcttggaaacaatacttggaaatcaccggaaacaccatatgcggcgaaaagccaattagtgtgatactaagtgctttatcgaaaatccgtgatgccggtccttcaggcatcaaatttcagtggcctaattattcacagagttctcatgtgacaagtattgatgatagtagtgtcagttatgcttcaggttatgttactataggataatgatcacggctaaaacggtcgaatgtaagcatatatctttcgattgtataattgttcccaaatactacagcatctcaaggaaaaaaaaacaaaaacttccaaaaaaatcgaatccctgaggaatctttaatacattttcaatctatttaagttttataaacgtgtatatgagatgtcatgagcatgaattattaataataaaaactaaatcattaaagtaacttaaggagttaaagcccgggctttaattgttagcagccttgacttgagcaatcaattctggaacaaccttgttgacatcaccgacaatggccaaatcagcaaccttcatgattggagcttcgacatctttgttgatggcaatgatgtagtcagagtcttgcataccagccaagtgttggatggcaccagagataccacaagcaatgtacaaagttggtctgacggtcttaccggtttgaccgacttgcaagtccttgtcaacccattccttttcaatggcagctctggaagcagcaatggtaccacccaacaaagaagctaattcttccaatttttcgaagttttccttggaaccaacaccacgaccaccagcaaccaaaaccttggcttcaccgatatcagcaatgtccttggccaatttgacaaccttggaaaccttggttctgatatcagaagcagtcaatttgatggcaaccttttcgatcttgtcatcagaaacgttagcatcgttaactggcaatttttcaaagacacctggtctgacggtggccatttgaggtctgtggtcagaacagacaatggtagcaatcaagttaccaccgaaagctggtctggtagccaacaagtcacggttttcgacatcgatatccaaagaggtacagtcagcagtcaaaccagtagacaatctggcagcaattcttggacccaagtctctaccgatgaaagtagcaccgatgaataagatttctggctttctttcgttgaccaagtcacagataaccttggcgtaaccgtcagtggagaaatgagctaataattcgttgtcagcagccaaaaccttgtcagcaccgtgggacaacaagtccttggacatcttttcagtgttgtgacccaataagacagcagtcaattcaacacccaatttttcagccatttccttacccttacctagcaattccaaagaaaccttttgtaattcaccatctctttgttcagcgaaaacccagacacccttgtagtcagccttgttcatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaggccggccgtttaaacgggccgccaccgcggtggagcctgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgctcgagtcgactgcagtttactgcttgtagtcgtaagaagtttggatgatatccttgatttcagaaatcaaagcttcctttgggttggcagtggtacattggtcttcgaaagccaattcagccattctgtcaatggattcgttcaattcttcttcagagacaccttgagatttcaagttcatttcaataccaacagattgacctaattcgtagacagccttggccaaagattcaaccaaagcttcagtggtgttacctttcaaacctaagaacttggcgatatcagcgtaatcggtgtcagctctgaagaattcgtactttgggaacaaagcgtgcttttgagggtccttggcgttgtatctgatgatgtgaggcaacaagatggcgttagctctaccatgtggaataccgtattcaccaccaattttgtgagcaatggagtgagcaatacccaagaaagcgttagcaaaggccataccggccaaagtagaagcgttgtgcatcttttctctggaaaccttgtcacctttttcaacggaagatttcaagtattcaaaggtcaatttgatagcttgtagggacaaacctctggtgtaatcggaggccatgacagaaacgtaagattccatagcgtgagtcaaaacgtccataccggtatcagcagtgacagattttgggacggacatgacaaattgagggtcaatgatggcgacatctggagtcaaagcgaaatcagccaatgggtatttgacgttggtttcagagtcagtgataacagcaaatggagtgacttcagaaccagtaccagaagtggttggaatacagatgaaagtggcgttttctggcataccaatcttgtaggttctcttaccaatgtccaagaatttttgcttggcaccgaagaaagaagtttctggatgttcgaagaacatccacatagccttggcagcatccatggcagaaccaccacccaaagcaatgatggtgtctggttggaaatcgaccatcatttccaaacccttgtagacagtgttggtggatggatttggttcaacttcagagaagatcttgatttgaggttgttcagttctttgacgtaagacgttttcgacggttttggtgtaaccaaattcaaccatacctgggtcacaaacgatcatgaccttttcaatcttgtccatggtggtcaaggacatgatagcgttttcttcgaaatagatttgagctggaaccttgaagatttgagtgttgtttcttctcttggcaatggtcttgatgttcaacaaatcggtagcagagacgttgtgggagatggagtttctaccgtaagaaccacaacccaaagtcaaggatggaatcaattcgttgtacatgtcaccgataccaccaacagcagatggagtgttgaccaaaacacgacaagccttcattcttagaccgaaatccttttgcaaagtttcgtcttcagtgtggataacagcagtgtgacctaaaccaccgaagtgcaaagtgtcttcacagatttggaaagcttgcttggtagattgagccttgactaaagccaaaactggagacaacttttctctggacaatgggtagtcagaaccgacaccggagatttcagcaatgatcaatttggtgttttctggaactgggataccggccaattcagcaatttcaacagcagacttaccaacgatatctggcttgataccagtcttttgttcgttcatgatggcgttttctaatctttgtaattcgtccttcttgacgaagtaagcttggtgagccttgaattcattggtgacatccttgtagatttccttgtcaatgacaacaacttgttcagaagcacagatcataccattatcgaaagtcttggaaccgatgatatcgttgacagcacgcttgatatgagcagtcttttcgatgtaagatggaacgttacctggaccaacacccaaagctggtttaccagtggagtaagcagacttaaccataccagaaccaccggtagccaagactaaagcaatacccttgtggttcatcaattgcttggtagcttcaatggatggaacttcaatccattggatgatatcctttggagcaccagccttcatggcagcttccaaaacaacttcagcagctctcttggaagattcttgagcagatgggtggaaagcgaaaataattgggttaccagtcttgatggcaatcatagccttgaagatggtggtagaagttgggttggtggttggagtgacaccacagatgacaccaattggttcagcaacgtaggtcaaacccttttctttgtcttcaccaatgataccaacagtcttgttgtccttgatggagttccagatgtattcagaggcgtataagttcttgatagccttgtcttcgtagatacctctaccggtttcttcatgagccaacttggccaaaaccatgtgttggtcaacagcagccaaggacatttggtggacaatgtggtcaatttcttcttgagacttcttggacaaagcttccaaagccttcttacctttgtcagctaaagcatcaatcatgatggcaacttcttgttccttggaacctctgttttccttttctggaatggtcaacattttttactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 45 - DNA, 11180 bp, synthetic construct
  misc_feature 1..11180: note = pBOL116
  source 1..11180: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccatttaaacggccggccctagatcagagggtggtaaatgaagtgtaatagtattcatttttcttataaatcatcccttccgtgatttatacaaaagaagaggagaatatgctgaatacttggtatattactctacattatactcttatcttgacgggtattctgagcatcttactcagtttcaagatcttttaatgtccaaaaacatttgagccgatctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaaatgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaaagcccgggcggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgtttaaatgtttaaacatggacagatatgcgatgaaaacgctaagtgatactccaaatggtgaaaggtacgatgcttggaaacaatacttggaaatcaccggaaacaccatatgcggcgaaaagccaattagtgtgatactaagtgctttatcgaaaatccgtgatgccggtccttcaggcatcaaatttcagtggcctaattattcacagagttctcatgtgacaagtattgatgatagtagtgtcagttatgcttcaggttatgttactataggataatgatcacggctaaaacggtcgaatgtaagcatatatctttcgattgtataattgttcccaaatactacagcatctcaaggaaaaaaaaacaaaaacttccaaaaaaatcgaatccctgaggaatctttaatacattttcaatctatttaagttttataaacgtgtatatgagatgtcatgagcatgaattattaataataaaaactaaatcattaaagtaacttaaggagttaaagcccgggctttaattgttagcagccttgacttgagcaatcaattctggaacaaccttgttgacatcaccgacaatggccaaatcagcaaccttcatgattggagcttcgacatctttgttgatggcaatgatgtagtcagagtcttgcataccagccaagtgttggatggcaccagagataccacaagcaatgtacaaagttggtctgacggtcttaccggtttgaccgacttgcaagtccttgtcaacccattccttttcaatggcagctctggaagcagcaatggtaccacccaacaaagaagctaattcttccaatttttcgaagttttccttggaaccaacaccacgaccaccagcaaccaaaaccttggcttcaccgatatcagcaatgtccttggccaatttgacaaccttggaaaccttggttctgatatcagaagcagtcaatttgatggcaaccttttcgatcttgtcatcagaaacgttagcatcgttaactggcaatttttcaaagacacctggtctgacggtggccatttgaggtctgtggtcagaacagacaatggtagcaatcaagttaccaccgaaagctggtctggtagccaacaagtcacggttttcgacatcgatatccaaagaggtacagtcagcagtcaaaccagtagacaatctggcagcaattcttggacccaagtctctaccgatgaaagtagcaccgatgaataagatttctggctttctttcgttgaccaagtcacagataaccttggcgtaaccgtcagtggagaaatgagctaataattcgttgtcagcagccaaaaccttgtcagcaccgtgggacaacaagtccttggacatcttttcagtgttgtgacccaataagacagcagtcaattcaacacccaatttttcagccatttccttacccttacctagcaattccaaagaaaccttttgtaattcaccatctctttgttcagcgaaaacccagacacccttgtagtcagccttgttcatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaggccggccgtttaaacgggccgccaccgcggtggagcctgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgctcgagtcgactgcagtttacaagttcaatttggccatgatggccttaacaatggcttgaacatcttcgttgtcttctggttcagctggagcagaagaagaagcagcaccgacaccgaaagcttctctgatttcttcgacggtggtggtaccgtaagcaacctttctgatgttgaacaagttttctggaccaacgttgtcagaggtggcagaaccaccaacagcaccacaacctaaagtcaaagatgggactaagttggtagcaccaccgataccacccaaagaacctggagagttaaccaaaattctggaaacaggcttcttcaaagcaaattctctaatgatttcttcgttttgagagtggatgatcaaagtgtgaccagaaccttggttgtgcaataaagccaaagacttttcacaagcttcatgccagtcttcgacggtgtagaaagccaagactggagccaatttttccttagcgtatggatttttaggagaaacatcggtttgttcggatagtaagataacagcatcagatggaatggaaataccagccaatttggccaaagcttggacatccttaccaacgatggctgggtttggagtaccgttggcacgtaatagaatcttaccaaccttttcagattcttcagcattcaagaagtaacccttttgtctcttgaattcttcgatgatttcagccttcttgacggtttcagcaatgatggattgttcagaagcacagatgacaccgttgtcgaaagtgtcagaaccgataacctttctaacagcagttggaatgtcagcagttctttcgatgaaacatggaccgttacctggaccgacaccgatggctggagtaccagaggagtaagcagctctaaccataccttcaccaccggtagccaagatcaaagcggtgtccttgttcttcatcaattcagcagtaccttcaacggtcaaaatggacatacattggatcaaaccatctggagcaccagcttcaacagcagccttttgcatgatcttaacggtttcagtgatggaacggacagcagttgggtgtggagagaagacaatggcgttaccagccttcaaagcaatcaagaccttgaaaatggcagtggaagttgggttggtagatggaatcaaaccagcaatgacacctaatgggacagcaatgtcaatcaatttcttttccttgtcttccttcaagataccaacggtcttcaaatccttgatgtagttgtagacaacaatggaggagaatttgttcttgatgaccttgtcttcccatttaccgtaaccggtgtcttcgtaagccaatttggccaatttgacagcttcaacttcagtagccttggcgatcttttcgatgaccttgttaacagcttcttgggaaaagttcttgaattcagcttgagccttcttggccttggcaatcaaagttctaacttcttggatggattgcaaatccttgtccatgatttccattttttactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 11108 bp, synthetic construct
  misc_feature 1..11108: note = pBOL118
  source 1..11108: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccatttaaacggccggccctagatcagagggtggtaaatgaagtgtaatagtattcatttttcttataaatcatcccttccgtgatttatacaaaagaagaggagaatatgctgaatacttggtatattactctacattatactcttatcttgacgggtattctgagcatcttactcagtttcaagatcttttaatgtccaaaaacatttgagccgatctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaaatgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaaagcccgggcggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgtttaaatgtttaaacatggacagatatgcgatgaaaacgctaagtgatactccaaatggtgaaaggtacgatgcttggaaacaatacttggaaatcaccggaaacaccatatgcggcgaaaagccaattagtgtgatactaagtgctttatcgaaaatccgtgatgccggtccttcaggcatcaaatttcagtggcctaattattcacagagttctcatgtgacaagtattgatgatagtagtgtcagttatgcttcaggttatgttactataggataatgatcacggctaaaacggtcgaatgtaagcatatatctttcgattgtataattgttcccaaatactacagcatctcaaggaaaaaaaaacaaaaacttccaaaaaaatcgaatccctgaggaatctttaatacattttcaatctatttaagttttataaacgtgtatatgagatgtcatgagcatgaattattaataataaaaactaaatcattaaagtaacttaaggagttaaagcccgggctttaattgttagcagccttgacttgagcaatcaattctggaacaaccttgttgacatcaccgacaatggccaaatcagcaaccttcatgattggagcttcgacatctttgttgatggcaatgatgtagtcagagtcttgcataccagccaagtgttggatggcaccagagataccacaagcaatgtacaaagttggtctgacggtcttaccggtttgaccgacttgcaagtccttgtcaacccattccttttcaatggcagctctggaagcagcaatggtaccacccaacaaagaagctaattcttccaatttttcgaagttttccttggaaccaacaccacgaccaccagcaaccaaaaccttggcttcaccgatatcagcaatgtccttggccaatttgacaaccttggaaaccttggttctgatatcagaagcagtcaatttgatggcaaccttttcgatcttgtcatcagaaacgttagcatcgttaactggcaatttttcaaagacacctggtctgacggtggccatttgaggtctgtggtcagaacagacaatggtagcaatcaagttaccaccgaaagctggtctggtagccaacaagtcacggttttcgacatcgatatccaaagaggtacagtcagcagtcaaaccagtagacaatctggcagcaattcttggacccaagtctctaccgatgaaagtagcaccgatgaataagatttctggctttctttcgttgaccaagtcacagataaccttggcgtaaccgtcagtggagaaatgagctaataattcgttgtcagcagccaaaaccttgtcagcaccgtgggacaacaagtccttggacatcttttcagtgttgtgacccaataagacagcagtcaattcaacacccaatttttcagccatttccttacccttacctagcaattccaaagaaaccttttgtaattcaccatctctttgttcagcgaaaacccagacacccttgtagtcagccttgttcatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaggccggccgtttaaacgggccgccaccgcggtggagcctgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgctcgagtcgactgcagtttaaacaattctgaaagcatcgaccaaaacacaacgacgtaatctgacgaaagttctggcagaagtgacaccttcaccagttggggtggtgatggtcatggtggtccaaccttcaccacccaaacccaaaccagcgatacatggaccgttcttgacaaagatggaagtgtcaatggcgttagccatttggttcatgttttcgatgtttctggagtgcatggcagcagtgtggtgacaaccaccttccaatttgacagccaaagcaatagcgtcagcaacgttagcaacacggacaactggtaagactggcatcatcaattcagtgacagcaaatgggtgttcagcggtggtttcgacgaataataatctggtttcttgtggaaccttcaaaccgatggcagcagcaatcttaccagcatctctaccaacccagtctctggagacggtacccttacctctttcatcgatgttcttcaacaaaactggttgcaattgttgagcttgttcagcagtcaacttgacggcatgttgaccttccatcaatctcatcaattcgtcagcaacggagtcaacaacaatcaaaaccttttcgtcagcacagatgatgttgttgtcgaaagaagcacccttgacaatggattgagcagctctggccaaatcagcggtttcatcgacaacaacaggagggttaccagcaccagcagcaatcaatctcttgttggtgtgctttctggcagcttcaacaacagcttcaccaccagtgacgactaatagaccgatacctgggaacttgaataatctttgagcagtttcgatatctgggttggcaacagtgaccaacaagttttctggaccaccagcagcaacaatggcttggttcaatagagtgatggctctttgagaaaccttcttggcagctgggtgtggagcgaagataacggagttaccagcagcaatcaaagagatggcgttgttgatgacagtagcagctgggttggtagatggggtgacggaagcaacaacaccccatggagcattttcaatcaaagtcaaaccattatcaccggtcaagacttgtggagacaaacattcgacacctggagtacctctagcttgagcaacgttcttagcgaatttgtcttcaactctacccataccggtttcggagacagccaattcagccaagtctctggcatgcttttcaccagcttctctgatggcagcaatggccaattgtctcatggcaacagatttcaaaccttgttgagcaaccttggcagcagcaacagcgtcgtccaaagaagcgaaaacacccatttcgtggacagcagcagatggagtgtcagaagattgcattttcaacaagacagccttgacaacttgttcgatatcttgttggttcattttttactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 47 - DNA, 11114 bp, synthetic construct
  misc_feature 1..11114: note = pBOL120
  source 1..11114: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggcccatttaaacggccggccctagatcagagggtggtaaatgaagtgtaatagtattcatttttcttataaatcatcccttccgtgatttatacaaaagaagaggagaatatgctgaatacttggtatattactctacattatactcttatcttgacgggtattctgagcatcttactcagtttcaagatcttttaatgtccaaaaacatttgagccgatctaaatacttctgtgttttcattaatttataaattgtactcttttaagacatggaaagtaccaacatcggttgaaacagtttttcatttacttatggtttattggtttttccagtgaatgattatttgtcgttaccctttcgtaaaagttcaaacacgtttttaagtattgtttagttgctctttcgacatatatgattatccctgcgcggctaaagttaaggatgcaaaaaacataagacaactgaagttaatttacgtcaattaagttttccagggtaatgatgttttgggcttccactaattcaataagtatgtcatgaaatacgttgtgaagagcatccagaaataatgaaaagaaacaacgaaactgggtcggcctgttgtttcttttctttaccacgtgatctgcggcatttacaggaagtcgcgcgttttgcgcagttgttgcaacgcagctacggctaacaaagcctagtggaactcgactgatgtgttagggcctaaaactggtggtgacagctgaagtgaactattcaatccaatcatgtcatggctgtcacaaagaccttgcggaccgcacgtacgaacacatacgtatgctaatatgtgttttgatagtacccagtgatcgcagacctgcaatttttttgtaggtttggaagaatatataaaggttgcactcattcaagatagtttttttcttgtgtgtctattcattttattattgtttgtttaaatgttaaaaaaaccaagaacttagtttcaaattaaattcatcacacaaacaaacaaaacaaaatgaacattgttgtttgtttgaagcaagttccagacactgctgaagtcagaattgacccagtcaagggtactttaatcagagaaggtgttccatctatcatcaacccagacgacaagaacgctttggaagaagctttggttttgaaggacaactacggtgctcacgttaccgtcatttccatgggtccacctcaagccaagaacgctttggttgaagctttggccatgggtgctgatgaagctgtcttattgactgacagagctttcggtggtgctgatactttagctacctctcacaccattgctgctggtatcaagaaattgaaatacgatatcgtctttgccggtcgtcaagccatcgatggtgataccgctcaagtcggtccagaaattgctgaacatttgggtattccacaagtcacctacgttgaaaaggttgaagttgacggtgacactttgaagatcagaaaggcttgggaagacggttacgaagttgttgaagtcaagactccagttctattgactgccatcaaggaattgaacgttccaagatacatgtccgttgaaaagatcttcggtgctttcgacaaggaagtcaagatgtggactgctgatgatatcgatgtcgacaaggccaacttgggtttgaaaggttctccaaccaaggtcaagaaatcttctaccaaggaagtcaagggtcaaggtgaagtcattgacaaaccagtcaaggaagctgccgcttacgttgtttccaagttgaaggaagaacactacatctaaagcccgggcggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatgtttaaatgtttaaacatggacagatatgcgatgaaaacgctaagtgatactccaaatggtgaaaggtacgatgcttggaaacaatacttggaaatcaccggaaacaccatatgcggcgaaaagccaattagtgtgatactaagtgctttatcgaaaatccgtgatgccggtccttcaggcatcaaatttcagtggcctaattattcacagagttctcatgtgacaagtattgatgatagtagtgtcagttatgcttcaggttatgttactataggataatgatcacggctaaaacggtcgaatgtaagcatatatctttcgattgtataattgttcccaaatactacagcatctcaaggaaaaaaaaacaaaaacttccaaaaaaatcgaatccctgaggaatctttaatacattttcaatctatttaagttttataaacgtgtatatgagatgtcatgagcatgaattattaataataaaaactaaatcattaaagtaacttaaggagttaaagcccgggctttaattgttagcagccttgacttgagcaatcaattctggaacaaccttgttgacatcaccgacaatggccaaatcagcaaccttcatgattggagcttcgacatctttgttgatggcaatgatgtagtcagagtcttgcataccagccaagtgttggatggcaccagagataccacaagcaatgtacaaagttggtctgacggtcttaccggtttgaccgacttgcaagtccttgtcaacccattccttttcaatggcagctctggaagcagcaatggtaccacccaacaaagaagctaattcttccaatttttcgaagttttccttggaaccaacaccacgaccaccagcaaccaaaaccttggcttcaccgatatcagcaatgtccttggccaatttgacaaccttggaaaccttggttctgatatcagaagcagtcaatttgatggcaaccttttcgatcttgtcatcagaaacgttagcatcgttaactggcaatttttcaaagacacctggtctgacggtggccatttgaggtctgtggtcagaacagacaatggtagcaatcaagttaccaccgaaagctggtctggtagccaacaagtcacggttttcgacatcgatatccaaagaggtacagtcagcagtcaaaccagtagacaatctggcagcaattcttggacccaagtctctaccgatgaaagtagcaccgatgaataagatttctggctttctttcgttgaccaagtcacagataaccttggcgtaaccgtcagtggagaaatgagctaataattcgttgtcagcagccaaaaccttgtcagcaccgtgggacaacaagtccttggacatcttttcagtgttgtgacccaataagacagcagtcaattcaacacccaatttttcagccatttccttacccttacctagcaattccaaagaaaccttttgtaattcaccatctctttgttcagcgaaaacccagacacccttgtagtcagccttgttcatgtttagttaattatagttcgttgaccgtatattctaaaaacaagtactccttaaaaaaaaaccttgaagggaataaacaagtagaatagatagagagaaaaatagaaaatgcaagagaatttatatattagaaagagagaaagaaaaatggaaaaaaaaaaataggaaaagccagaaatagcactagaaggagcgacaccagaaaagaaggtgatggaaccaatttagctatatatagttaactaccggctcgatcatctctgcctccagcatagtcgaagaagaattttttttttcttgaggcttctgtcagcaactcgtattttttctttcttttttggtgagcctaaaaagttcccacgttctcttgtacgacgccgtcacaaacaaccttatgggtaatttgtcgcggtctgggtgtataaatgtgtgggtgcaggccggccgtttaaacgggccgccaccgcggtggagcctgtgtggaagaacgattacaacaggtgttgtcctctgaggacataaaatacacaccgagattcatcaactcattgctggagttagcatatctacaattgggtgaaatggggagcgatttgcaggcatttgctcggcatgccggtagaggtgtggtcaataagagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgctcgagtcgactgcagtttatctaatggagaaaccatcagtcaagacacatcttcttcttctagcgaagtgacgggcagtggtagtaccttcaccagttggagtagcaatggtgaaagtggtggaaccttcacctctgaaacctaaaccagcgaaagatggaccgttcttgacaaagatggaggtttgcatgtcacgggcagccttgttcaatctggagatgttttgagagtgcatggtagcagtgtgatgtagaccttgttccaattcaatggcaacttccaaagcttcatcgaagtctggaactctgacaactggaacaattggcatcaacaattcaacagtagcgaatgggtgggacttttcagtttcgacaatgatcaatcttggggtgaaatcacaagcaataccagcttctttcaagatttcagtggcagacttaccaaccaatttcttgttggtgacacccttgtcagtgacggcaaccttttccaatttttggatatcagatgggttagtgacgtgcaaagcaccgttcttttccatttggaacaacaagaagtcagcaatggagtcaacggcaacaacagacttttcagcgatacacaagatattatggtcaaaggaagcaccgtcgacaatgtcagcagcagccttttcaatgttagcggtttcgtcaacgatggatggagggttaccagcaccagcaccgataaccttcttaccagattgcatagcttgcaagacaacacctggaccaccagtgatgaccaacaatggaacctttgggtggttcatcatttcttgagcagcttggatagatggcttggcaacggtgacaatcaagttgtcaataccacaagaatctctgacgatagtgttcaacttttcaatcaaccataaagagatgttcttggcacctgggtgaggagagtagaaaacggcgttaccagcagccaacataccgatggagttacagatcaaagtttcagttgggttggtagatggagcaacagcaccgatgacaccgtatggagataattcgtataaagtcataccgttgtcaccggtagcaacttcagtgtacaagtcttcaacacctggagtcttttcgatagctaaagtgttcttcaagattttatcggtgacattacccataccggtttcagcaacagctctggtagcaatggtttcgatttctgggtataaagcttctctgatggccttgacaacgtttcttctttcttccaaagatttttccttgtaacagttttgagcaatgacggcagcttggacagcttcatcgacggtatcgaaaacaccggacttggcaccttgggtggtggtcttggttggaacttccttttgttcagccaatttttccaacaaaaccttcttgactaattgttccaattccaaagattccattttttactagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcgagtattgataatgataaactgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 48 - DNA, 2613 bp, Entamoeba histolytica
  source 1..2613: mol_type = other DNA
atgtcaacacaacaaactatgactgtagatgaacatattaatcaacttgttgctaaagcacaagttgcacttaaagaatatcttaaaccagaatatacacaagaaaaaatagattatattgtaaagaaagcatcagttgcagcacttgatcaacattgtgcacttgcagcagctgcagttgaagaaacaggaagaggtatttttgaagataaagctactaaaaatatatttgcatgtgaacatgttacacatgaaatgagacatgctaaaacagttggtattattaatgtagatccactttatggaattacagaaattgcagaaccagttggagttgtttgtggagttacaccagttactaatccaacatcaacagctattttcaagtcacttatttcaattaaaacaagaaatccaattgtattttcattccatccatcagcacttaaatgttctattatggcagctaaaattgttagagatgcagctattgcagcaggagcaccagaaaattgtattcaatggattgaatttggaggaattgaagcatcaaataaattaatgaatcatccaggagttgctactattcttgctacaggaggaaatgctatggttaaagcagcatattcatcaggaaaaccagcacttggagtaggagcaggaaatgtaccaacatatattgaaaaaacatgtaatattaaacaagcagcaaatgatgtagttatgtcaaaatcatttgataatggtatgatttgtgcatcagaacaagcagcaattattgataaagaaatttatgatcaagtagttgaagaaatgaaaacacttggagcatatttcattaatgaagaagaaaaagctaaattagaaaagtttatgtttggagttaatgcatattcagcagatgttaataatgcaagacttaatccaaaatgtccaggtatgtcaccacaatggtttgctgaacaagttggaattaaagttccagaagattgtaatattatttgtgcagtttgtaaagaagttggaccaaatgaaccattaacaagagaaaaattatcaccagttcttgctattcttaaagcagaaaatacacaagatggtattgataaagctgaagctatggttgaatttaatggtagaggacattcagcagctattcattcgaatgataaagcagtagttgaaaagtatgcacttacaatgaaagcatgcagaattttacataatacaccatcatcacaaggaggaattggatcaatttataactatatttggccatcatttacacttggatgtggatcatatggaggaaattcggtatcagctaatgttacatatcataatttattaaatattaaaagacttgcagatagaagaaacaaccttcaatggttcagagttccaccaaagattttctttgaaccacattctattagatatcttgctgaacttaaggaacttagtaaaatattcattgtttcagatagaatgatgtataaattaggatatgtagatagagttatggatgtattgaaaagaagaagtaatgaagtagaaattgaaattttcattgatgtagaaccagatccatctattcaaaccgttcaaaaaggacttgctgttatgaatacatttggaccagataatattattgctattggaggaggatcagctatggatgcagctaagattatgtggttactttatgaacatccagaagccgatttctttgcaatgaaacaaaaattcattgatcttagaaagagagcatttaaattcccaacaatgggtaagaaagctagattaatttgtattccaacaacatcaggaactggatcagaagttacaccatttgcagttatttcagatcatgaaacaggtaagaaatatccacttgctgattattcacttacaccatcagttgctattgttgatccaatgtttactatgtcacttccaaagagagctattgctgatactggacttgatgtattggttcatgcaacagaagcatatgtttcagttatggctaatgaatatactgatggacttgctagagaagcagttaaattagtctttgaaaatcttcttaaatcatataatggagatttagaagcaagagaaaagatgcacaatgctgcaacaattgcaggtatggcatttgcatcagcattccttggtatggaccattccatggcacataaagttggagcagcattccatcttccacatggtagatgtgtagcagtattattaccacatgtcattagatataatggacaaaaaccaagaaagcttgcaatgtggccaaaatataatttctataaggcagaccaaagatatatggaacttgcacaaatggttggacttaaatgtaatacaccagctgaaggagttgaagcatttgctaaagcatgtgaagaattaatgaaagccacagagactattactggattcaagcaagcaaatattgatgaagcagcatggatgagtaaagtaccagaaatggcacttcttgcatttgaagatcaatgttcaccagctaatccaagagtcccaatggttaaggatatggaaaagattctcaaagctgcatattatccaattgcttga

SEQ ID NO: 49 - DNA, 2610 bp, synthetic construct
  misc_feature 1..2610: note = adh2 E. histolytica codon pair optimised
  source 1..2610: mol_type = other DNA
atgtccactcaacaaaccatgaccgttgatgaacacattaaccaattggtcagaaaggctcaagttgctttgaaggaatacttgaaaccagaatacactcaagaaaagatcgattacattgtcaagaaggcttctgttgctgctctagaccaacactgtgctttggctgctgctgctgtcgaagaaactggtcgtggtatctttgaagacaaagctaccaagaacattttcgcttgtgaacacgtcactcacgaaatgagacacgccaagaccgttggtatcatcaacgttgatccattatacggtatcactgaaattgctgaaccagtcggtgttgtctgtggtgtcaccccagttaccaacccaacttctactgccattttcaaatctttgatttccatcaagaccagaaacccaattgttttctccttccacccatctgctttgaaatgttccatcatggctgccaagatcgtcagagatgctgccattgctgctggtgctccagaaaactgtatccaatggatcgaatttggtggtattgaagcttccaacaaattgatgaaccatcctggtgttgctaccatcttagctactggtggtaacgctatggtcaaggctgcttactcttctggtaagccagctttgggtgtcggtgctggtaacgtcccaacttacatcgaaaagacctgtaatatcaagcaagctgctaacgatgttgtcatgtccaagtctttcgacaacggtatgatctgtgcctccgaacaagctgccatcatcgacaaagaaatctacgaccaagttgttgaagaaatgaagactttgggtgcttacttcatcaacgaagaagaaaaggccaaattggaaaaattcatgttcggtgttaatgcttactctgctgatgtcaacaacgccagattgaacccaaagtgtccaggtatgtctccacaatggttcgctgaacaagtcggtatcaaggttccagaagactgtaacatcatctgtgccgtttgtaaggaagttggtccaaacgaaccattgaccagagaaaagttgtctccagttttggccattttgaaggctgaaaacactcaagatggtattgacaaggctgaagctatggtcgaattcaacggtcgtggtcactctgctgccattcactccaatgacaaggctgttgttgaaaaatacgctttgaccatgaaggcttgtcgtatcttgcacaacactccatcttctcaaggtggtatcggttccatttacaactacatctggccatctttcactttaggttgtggttcttacggtggtaactccgtttctgccaatgttacctaccacaacttgttgaacatcaagagattggctgacagaagaaacaacttacaatggttcagagtcccaccaaagatcttcttcgaacctcactccattagatacttggctgaattgaaggaattgtccaagattttcattgtctctgacagaatgatgtacaaattgggttacgttgacagagttatggatgtcttgaagagaagatccaacgaagttgaaattgaaatcttcatcgatgttgaaccagacccatccattcaaaccgtccaaaagggtttggctgtcatgaacactttcggtccagacaacatcattgccattggtggtggttctgccatggatgctgccaagatcatgtggttattatacgaacatccagaagctgatttcttcgctatgaagcaaaaattcatcgatttaagaaagagagctttcaagttcccaaccatgggtaagaaggccagattaatctgtatcccaaccacttctggtaccggttctgaagtcaccccattcgctgtcatctctgaccacgaaactggtaagaagtatccattggctgactactctttgaccccatccgttgccattgttgacccaatgtttaccatgtccttgcctaagagagccattgctgacactggtttggatgtcttagtccacgctactgaagcttacgtttctgttatggctaacgaatacactgacggtttggccagagaagctgtcaaattggttttcgaaaacttgttgaaatcttacaacggtgacttggaagctcgtgaaaagatgcacaacgctgctaccattgctggtatggcctttgcttctgctttcttgggtatggaccattccatggctcacaaggtcggtgctgctttccatttgccacacggtagatgtgttgccgttttgttgcctcacgttatcagatacaacggtcaaaagccaagaaagttggccatgtggccaaagtacaacttctacaaggctgatcaaagatacatggaattggctcaaatggtcggtttgaagtgtaacaccccagctgaaggtgtcgaagcctttgccaaggcttgtgaagaattgatgaaggctactgaaaccatcactggtttcaagaaggccaacattgatgaagctgcttggatgtccaaggttccagaaatggctctattggctttcgaagaccaatgttctccagctaacccaagagtcccaatggttaaggacatggaaaagattttgaaggctgcttactacccaatcgct

SEQ ID NO: 50 - protein, 870 aa, Entamoeba histolytica
MSTQQTMTVDEHINQLVRKAQVALKEYLKPEYTQEKIDYIVKKASVAALDQHCALAAAAVEETGRGIFEDKATKNIFACEHVTHEMRHAKTVGIINVDPLYGITEIAEPVGVVCGVTPVTNPTSTAIFKSLISIKTRNPIVFSFHPSALKCSIMAAKIVRDAAIAAGAPENCIQWIEFGGIEASNKLMNHPGVATILATGGNAMVKAAYSSGKPALGVGAGNVPTYIEKTCNIKQAANDVVMSKSFDNGMICASEQAAIIDKEIYDQVVEEMKTLGAYFINEEEKAKLEKFMFGVNAYSADVNNARLNPKCPGMSPQWFAEQVGIKVPEDCNIICAVCKEVGPNEPLTREKLSPVLAILKAENTQDGIDKAEAMVEFNGRGHSAAIHSNDKAVVEKYALTMKACRILHNTPSSQGGIGSIYNYIWPSFTLGCGSYGGNSVSANVTYHNLLNIKRLADRRNNLQWFRVPPKIFFEPHSIRYLAELKELSKIFIVSDRMMYKLGYVDRVMDVLKRRSNEVEIEIFIDVEPDPSIQTVQKGLAVMNTFGPDNIIAIGGGSAMDAAKIMWLLYEHPEADFFAMKQKFIDLRKRAFKFPTMGKKARLICIPTTSGTGSEVTPFAVISDHETGKKYPLADYSLTPSVAIVDPMFTMSLPKRAIADTGLDVLVHATEAYVSVMANEYTDGLAREAVKLVFENLLKSYNGDLEAREKMHNAATIAGMAFASAFLGMDHSMAHKVGAAFHLPHGRCVAVLLPHVIRYNGQKPRKLAMWPKYNFYKADQRYMELAQMVGLKCNTPAEGVEAFAKACEELMKATETITGFKKANIDEAAWMSKVPEMALLAFEDQCSPANPRVPMVKDMEKILKAAYYPIA

SEQ ID NO: 51 - DNA, 2658 bp, Piromyces sp.
  source 1..2658: mol_type = other DNA; note = E2
atgtccggattacaaatgttccaaaacctttctctttacggtagtctcgccgaaatcgatactagcgaaaagcttaacgaagctatggacaaattaactgctgcccaagaacaattcagagaatacaaccaagaacaagttgacaaaatcttcaaggctgttgctttagctgcttctcaaaaccgtgttgctttcgctaagtacgcacacgaagaaacccaaaagggtgttttcgaagataaggttatcaagaacgaattcgctgctgattacatttaccacaagtactgcaatgacaagaccgccggtatcattgaatatgatgaagccaatggtcttatggaaattgctgaaccagttggtccagttgttggtattgctccagttactaacccaacttctactatcatctacaagtctttaattgccttaaagacccgtaactgtattatcttctcaccacatccaggagctcacaaggcctctgttttcgttgttaaggtcttacaccaagctgctgttaaggctggtgccccagaaaactgtattcaaatcatcttcccaaagatggatttaactactgaattattacaccaccaaaagactcgtttcatttgggctactggtggtccaggtttagttcacgcctcttacacttctggtaagccagctcttggtggtggtccaggtaatgctccagctcttattgatgaaacttgtgatatgaacgaagctgttggttctatcgttgtttctaagactttcgattgtggtatgatctgtgccactgaaaacgctgttgtcgttgtcgaatctgtctacgaaaacttcgttgctaccatgaagaagcgtggtgcctacttcatgactccagaagaaaccaagaaggcttctaaccttcttttcggagaaggtatgagattaaatgctaaggctgttggtcaaactgccaagactttagctgaaatggccggtttcgaagtcccagaaaacaccgttgttctctgtggtgaagcttctgaagttaaattcgaagaaccaatggctcacgaaaagttaactactatcctcggtatctacaaggctaaggactttgacgatggtgtcagattatgtaaggaattagttactttcggtggtaagggtcacactgctgttctctacaccaaccaaaacaacaaggaccgtattgaaaagtaccaaaacgaagttccagccttccacatcttagttgacatgccatcttccctcggttgtattggtgatatgtacaacttccgtcttgctccagctcttaccattacttgtggtactatgggtggtggttcctcctctgataacattggtccaaagcacttacttaacatcaagcgtgttggtatgagacgcgaaaacatgctttggttcaagattccaaagtctgtctacttcaagcgtgctatcctttctgaagctttatctgacttacgtgacacccacaagcgtgctatcattattaccgatagaactatgactatgttaggtcaaactgacaagatcattaaggcttgtgaaggtcatggtatggtctgcactgtctacgataaggttgtcccagatccaactatcaagtgtattatggaaggtgttaatgaaatgaacgtcttcaagccagatttagctattgctcttggtggtggttctgctatggatgccgctaagatgatgcgtttattctacgaatacccagaccaagacttacaagatattgctactcgtttcgtcgatatccgtaagcgtgttgttggttgtccaaagcttggtagacttattaagactcttgtctgtatcccaactacctctggtactggtgccgaagttactccattcgctgtcgttacctctgaagaaggtcgtaagtacccattagtcgactacgaacttactccagatatggctattgttgatccagaattcgctgttggtatgccaaagcgtttaacttcttggactggtattgatgctcttacccacgccattgaatcttacgtttctattatggctactgacttcactagaccatactctctccgtgctgttggtcttatcttcgaatccctttcccttgcttacaacaacggtaaggatattgaagctcgtgaaaagatgcacaatgcttctgctattgctggtatggcctttgccaacgctttccttggttgttgtcactctgttgctcaccaacttggttccgtctaccacattccacacggtcttgccaacgctttaatgctttctcacatcattaagtacaacgctactgactctccagttaagatgggtaccttcccacaatacaagtacccacaagctatgcgtcactacgctgaaattgctgaactcttattaccaccaactcaagttgttaagatgactgatgttgataaggttcaatacttaattgaccgtgttgaacaattaaaggctgacgttggtattccaaagtctattaaggaaactggaatggttactgaagaagacttcttcaacaaggttgaccaagttgctatcatggccttcgatgaccaatgtactggtgctaacccacgttacccattagtttctgaattaaaacaattaatgattgatgcctggaacggtgttgtcccaaagctctaa

SEQ ID NO: 52 - protein, 885 aa, Piromyces sp.
  source 1..885: note = E2
MSGLQMFQNLSLYGSLAEIDTSEKLNEAMDKLTAAQEQFREYNQEQVDKIFKAVALAASQNRVAFAKYAHEETQKGVFEDKVIKNEFAADYIYHKYCNDKTAGIIEYDEANGLMEIAEPVGPVVGIAPVTNPTSTIIYKSLIALKTRNCIIFSPHPGAHKASVFVVKVLHQAAVKAGAPENCIQIIFPKMDLTTELLHHQKTRFIWATGGPGLVHASYTSGKPALGGGPGNAPALIDETCDMNEAVGSIVVSKTFDCGMICATENAVVVVESVYENFVATMKKRGAYFMTPEETKKASNLLFGEGMRLNAKAVGQTAKTLAEMAGFEVPENTVVLCGEASEVKFEEPMAHEKLTTILGIYKAKDFDDGVRLCKELVTFGGKGHTAVLYTNQNNKDRIEKYQNEVPAFHILVDMPSSLGCIGDMYNFRLAPALTITCGTMGGGSSSDNIGPKHLLNIKRVGMRRENMLWFKIPKSVYFKRAILSEALSDLRDTHKRAIIITDRTMTMLGQTDKIIKACEGHGMVCTVYDKVVPDPTIKCIMEGVNEMNVFKPDLAIALGGGSAMDAAKMMRLFYEYPDQDLQDIATRFVDIRKRVVGCPKLGRLIKTLVCIPTTSGTGAEVTPFAVVTSEEGRKYPLVDYELTPDMAIVDPEFAVGMPKRLTSWTGIDALTHAIESYVSIMATDFTRPYSLRAVGLIFESLSLAYNNGKDIEAREKMHNASAIAGMAFANAFLGCCHSVAHQLGSVYHIPHGLANALMLSHIIKYNATDSPVKMGTFPQYKYPQAMRHYAEIAELLLPPTQVVKMTDVDKVQYLIDRVEQLKADVGIPKSIKETGMVTEEDFFNKVDQVAIMAFDDQCTGANPRYPLVSELKQLMIDAWNGVVPKL